(19) United States
(12) Patent Application Publication
(10) Pub. No.: US 2005/0120397 A1
(43) Pub. Date: Jun. 2, 2005

(54) Compounds and methods for regulation of spermatid differentiation

(76) Inventors: Hermann Steller, East Burke, VT (US); Eli Arama, New York, NY (US); Holger Kissel, New York, NY (US)

Correspondence Address: EITAN, PEARL, LATZER & COHEN ZEDEK LLP, 10 ROCKEFELLER PLAZA, SUITE 1001, NEW YORK, NY, 10020, US

(21) Appl. No.: 10/993,167

(22) Filed: Nov. 22, 2004

Related U.S. Application Data
(60) Provisional application No. 60/524,049, filed on Nov. 24, 2003.

(51) Int. Cl. A01K 67/00; A61K 38/05; C12N 5/08

(52) U.S. Cl. 800/8; 435/366; 435/325; 514/18

(57) ABSTRACT

The present invention provides methods for the regulation of spermatid differentiation. This invention also provides applications of regulated spermatid differentiation, including the treatment of infertility, contraception and pest control. This invention also describes agents for effecting such methods and compositions comprising same.

### CROSS REFERENCE TO RELATED APPLICATIONS

This application claims priority from U.S. Provisional Patent Application No. 60/524,049, filed Nov. 24, 2003, which is incorporated in its entirety herein.

### FIELD OF THE INVENTION

This invention provides methods for regulating spermatid differentiation. The present invention provides methods for stimulating, enhancing, inhibiting or diminishing caspase, cyt-c-d, Sept4/ARTS, and/or Bruce expression and/or activity in spermatocytes thereby regulating their differentiation to spermatids. The present invention also provides methods of use thereof, for applications in infertility, contraception and pest control.

### BACKGROUND OF THE INVENTION

Apoptosis is a morphologically distinct form of cell death that usually serves to remove unwanted and potentially dangerous cells. A key event during apoptosis is the activation of a specific class of cysteine proteases, termed caspases. Caspases are expressed as inactive zymogens in virtually all animal cells and are specifically activated in cells destined to undergo apoptosis. On the other hand, there are some isolated examples where apoptosis-like events do not lead to the death, but rather the terminal differentiation of certain cell types. For example, lens epithelial cells and mammalian red blood cells lose their nucleus and other subcellular organelles during terminal differentiation but continue to be metabolically active. Likewise, sperm cell terminal differentiation involves complex changes in the cytoarchitecture, some of which are reminiscent of apoptosis. Mutations in several genes associated with apoptosis produce defects in mouse spermatogenesis, in early stages of sperm development, but little is known about the role of apoptosis during sperm cell terminal differentiation.

Spermatogenesis in both flies and mammals begins in the male germ line with the appearance of stem cells that generate spermatogonia. Spermatogonia proliferate by a limited number of mitotic divisions to give rise to diploid, primary spermatocytes. Every primary spermatocyte undergoes meiotic divisions that result in four haploid, round spermatids. Postmeiotic sperm cell differentiation is characterized by a sequence of remarkably conserved changes in the morphology and the structural organization of the spermatids. In Drosophila, these changes include elongation of the flagellum, fusion and subsequent elongation of the mitochondria to the entire length of the spermatid, nuclear condensation and elongation, and the expelling of the bulk of the spermatid cytoplasm, a process described more than three decades ago, yet whose molecular and cellular regulation remain unknown.

### SUMMARY OF THE INVENTION

This invention provides, in one embodiment, methods for the regulation of spermatid differentiation. This invention provides, in other embodiments, applications of regulated spermatid differentiation, including the treatment of infertility, contraception and pest control.

In one embodiment, the invention provides a method of inhibiting or abrogating spermatid differentiation, comprising the step of contacting a spermatogenic cell with an agent that inhibits or abrogates caspase expression or activity in the cell, thereby inhibiting or abrogating spermatid differentiation.

The invention provides, in another embodiment, a method of inhibiting or abrogating spermatid differentiation, comprising the step of contacting a spermatogenic cell with an agent that inhibits or abrogates cyt-c-d expression or activity in said cell, thereby inhibiting or abrogating spermatid differentiation. In another embodiment, inhibiting or abrogating cyt-c-d expression or activity in the cell results in the inhibition or abrogation of caspase expression, activity and/or activation.

The invention provides, in another embodiment, a method of inhibiting or abrogating spermatid differentiation, comprising the step of contacting a spermatogenic cell with an agent that inhibits or abrogates Sept4/ARTS expression or activity in said cell, thereby inhibiting or abrogating spermatid differentiation. In another embodiment, inhibiting or abrogating Sept4/ARTS expression or activity in the cell results in the inhibition or abrogation of caspase expression, activity and/or activation.

The invention provides, in another embodiment, a method of inhibiting or abrogating spermatid differentiation, comprising the step of contacting a spermatogenic cell with an agent that inhibits or abrogates Bruce expression or activity in said cell, thereby inhibiting or abrogating spermatid differentiation. In another embodiment, inhibiting or abrogating Bruce expression or activity in the cell results in the inhibition or abrogation of caspase expression, activity and/or activation.

In another embodiment, the invention provides a method of male contraception in a subject, comprising contacting a spermatogenic cell in the subject with an agent that diminishes or abrogates caspase expression or activity in the spermatogenic cell. In another embodiment, the invention provides a method of male contraception in a subject, comprising contacting a spermatogenic cell in the subject with an agent that diminishes or abrogates cyt-c-d expression or activity in the spermatogenic cell. In another embodiment, the invention provides a method of male contraception in a subject, comprising contacting a spermatogenic cell in the subject with an agent that diminishes or abrogates Sept4/ARTS expression or activity in the spermatogenic cell. In another embodiment, the invention provides a method of male contraception in a subject, comprising contacting a spermatogenic cell in the subject with an agent that diminishes or abrogates Bruce expression or activity in the spermatogenic cell.

In another embodiment, there is provided a method of arthropod pest control, comprising, contacting a spermatogenic cell of said pest with an agent that diminishes or abrogates caspase expression or activity. The present invention provides, in another embodiment, a method of arthropod pest control, comprising, contacting a spermatogenic cell of said pest with an agent that diminishes or abrogates cyt-c-d expression or activity. The present invention provides, in another embodiment, a method of arthropod pest control, comprising, contacting a spermatogenic cell of said pest with an agent that diminishes or abrogates Sept4/ARTS expression or activity. The present invention provides, in another embodiment, a method of arthropod pest control, comprising, contacting a spermatogenic cell of said pest with an agent that diminishes or abrogates Bruce expression or activity. According to this aspect of the invention, in one embodiment, the arthropod is an insect.

The invention also provides a method of stimulating or enhancing spermatid differentiation, comprising the step of contacting a spermatogenic cell with an agent that stimulates or enhances caspase expression or activity in said cell, thereby stimulating or enhancing spermatid differentiation. In another embodiment, the invention provides a method of stimulating or enhancing spermatid differentiation, comprising the step of contacting a spermatogenic cell with an agent that stimulates or enhances cyt-c-d expression or activity in said cell, thereby stimulating or enhancing spermatid differentiation. In another embodiment, the invention provides a method of stimulating or enhancing spermatid differentiation, comprising the step of contacting a spermatogenic cell with an agent that stimulates or enhances Sept4/ARTS expression or activity in said cell, thereby stimulating or enhancing spermatid differentiation. In another embodiment, the invention provides a method of stimulating or enhancing spermatid differentiation, comprising the step of contacting a spermatogenic cell with an agent that stimulates or enhances Bruce expression or activity in said cell, thereby stimulating or enhancing spermatid differentiation.

In one embodiment, the invention provides a method of treatment of infertility in a male subject in need thereof, comprising contacting a spermatogenic cell in said subject with an agent that stimulates or enhances caspase expression or activity in said cell. In another embodiment, the invention provides a method of treatment of infertility in a male subject in need thereof, comprising contacting a spermatogenic cell in said subject with an agent that stimulates or enhances cyt-c-d gene expression in said cell. In another embodiment, the invention provides a method of treatment of infertility in a male subject in need thereof, comprising contacting a spermatogenic cell in said subject with an agent that stimulates or enhances Sept4/ARTS gene expression in said cell. In another embodiment, the invention provides a method of treatment of infertility in a male subject in need thereof, comprising contacting a spermatogenic cell in said subject with an agent that stimulates or enhances Bruce gene expression in said cell.

In another embodiment, the invention provides a method of identifying an agent that stimulates or increases the fertility rate of a male mammal comprising administering a candidate biologically active agent to a non-human male transgenic animal genetically disrupted for caspase, cyt-c-d, Sept4/ARTS, or Bruce gene function and determining the effect of said agent on a fertility rate of said non-human male transgenic animal, wherein an increased fertility rate indicates that the candidate biologically active agent increases the fertility rate of a mammal.

In another embodiment, the invention provides a method of identifying an agent that decreases or abrogates the fertility rate of a male mammal comprising administering a candidate biologically active agent to a non-human male animal expressing a caspase, cyt-c-d, Sept4/ARTS, or Bruce gene, wherein said agent alters said caspase, cyt-c-d, Sept4/ARTS, or Bruce gene or gene product expression or activity and determining the effect of said agent on a fertility rate of said non-human male animal, wherein a decreased fertility rate indicates that the candidate biologically active agent decreases or abrogates the fertility rate of a mammal. In another embodiment, the invention provides a method of identifying an agent that decreases or abrogates the fertility rate of a male arthropod comprising administering a candidate biologically active agent to a male arthropod expressing a caspase, cyt-c-d, Sept4/ARTS, or Bruce gene, wherein said agent alters said caspase, cyt-c-d, Sept4/ARTS, or Bruce gene or gene product expression or activity and determining the effect of said agent on a fertility rate of said male arthropod, wherein a decreased fertility rate indicates that the candidate biologically active agent decreases or abrogates the fertility rate of a male arthropod.

In another embodiment, the invention provides a method of monitoring in vivo caspase inhibition in a male subject in need, comprising the steps of obtaining a semen sample from a male subject in need and determining the presence of spermatid differentiation in said semen sample, wherein diminished spermatid differentiation or abnormal sperm morphology (e.g. cytoplamsic droplet sperm) indicates in vivo caspase inhibition.

### BRIEF DESCRIPTION OF THE DRAWINGS

FIG. 1 demonstrates phenotypic characteristics associated with spermatid individualization. A schematic diagram, (A) depicts a single cyst with four representative spermatids at increasingly advanced developmental stages (from top to bottom) of spermatid individualization. Structural components of the IC are demonstrated in panels (B)-(G), and CB and WB phenotypic correspondence with apoptosis, as assessed by acridine orange staining, is demonstrated in panels (H)-(J) (c.c., cyst cell; ind., post-individualized; preind., pre-individualized). Scale bars are equivalent to 50 μm.

FIG. 2 demonstrates the activation of the effector caspase, drICE at the onset of individualization (A) and its enlargement in (B). The assembly of the IC (phalloidin, red) at the nuclear end (DAPI, blue) of a cyst, which marks the beginning of the process of spermatid individualization, was followed by a steep gradient of drICE activation (CM1, green). Active drICE accumulated in the pre-individualized part of a cyst (arrows) and within the CB. Following caudal translocation of the CB, active drICE was no longer detectable in the post-individualized part of the spermatids (arrowheads). The activation of drICE during spermatid individualization was detected either by the anti-active mouse caspase 3 (CM1) antibody that crossreacts with drICE (C and D, green), or by the anti-active-drICE antibody (E and F, green). Both antibodies demonstrated intense staining in spermatid cytoplasm, namely the CB and distal parts of the cell. Once all the cytoplasm was extruded, active drICE is detectable only in the WB (C, elongated spermatid cysts. Scale bars, 50 μm).

FIG. 3 demonstrates that caspase activity inhibition prevents spermatid individualization. Normal testes cultured for 26 hours with the caspase inhibitor Z-VAD displayed flat, irregular CBs, indicating that the bulk cytoplasm was not collected properly in the CBs (B), as compared to controls taken from the same individual (A). In (C), translocation is visualized in control testes (nuclei were visualized with DAP1 (blue), ICs with phalloidin (red), and active-drICE with the CM1 antibody (green)) (note that only a few ICs remain associated with nuclei at the base of the testis). By contrast, testes cultured with Z-VAD displayed many early stages of IC assembly, but no evidence of IC translocation (D). Ectopic expression of the baculoviral broad-spectrum caspase inhibitor p35 blocked individualization (E), and revealed scattered IC cones and flat CBs and WBs A schematic representation of the last step of the individualization process in Drosophila and mammals (rat) highlights the overall similarity in sperm differentiation (F). Mammalian spermatids are also connected by cytoplasmic bridges, which are eliminated, together with most of the cytoplasm and organelles during terminal differentiation. The spermatid cytoplasm ends up in a structure named residual body (RB), which is comparable to the WB. Scanning electron microscopy (SEM) of a human spermatozoon displaying a cytoplasmic droplet (CD) morphological abnormality (G) exhibited a phenotype very similar to individualization defects seen in panels (B), (D), and (E) (Reproduced from Hollanders and Carver-Ward, 1996 with permission from Parthenon Publishing, UK [N, nucleus; WB, waste bag; RB, residual body. Scale bars in (A)-(E), 50 μm (in (F) 1 μm)].

FIG. 4, panels A-D demonstrates that caspase-3 is activated during mouse spermatid terminal differentiation. Paraffin sections of wild-type mouse testis were stained with CM1 antibody to detect active-caspase-3 (brown) and were then counterstained with hematoxylin (purple). The localization of the staining indicates that caspase 3 is activated at a very late stage during spermatogenesis, in the lumen of the seminiferous tubule. Specifically, strong staining was detected in the residual bodies (RB) that were pinched off from elongated spermatids into the lumen of the tubules (black arrows). Therefore, murine residual bodies share functional and biochemical similarities with the waste bag (WB) of Drosophila. Panel F depicts the cytoplasmic droplet morphology of sperm from the Sept4/ARTS homozygous mouse, as compared with wild type sperm morphology (E).

FIG. 5 demonstrates that caspase activation is independent of nuclear differentiation or IC assembly, since mutants that disrupt sperm differentiation retain CM1-staining. Purity of essence (poe¹) mutant spermatids contained significantly fewer ICs (arrowheads), and often the ICs were not detected at all (arrows) (A). In jaguar (jar¹) male flies, IC assembly and movement were impaired (B). Active-drICE (green) accumulated independently of functional IC assembly in jar¹ testes, but correlated with the differentiation stage of nuclei (blue). The black and white panels of nuclear bundles on the right correspond to the blue nuclei on the left; caspase activation was only detected in spermatid bundles with thin, fully differentiated nuclei (see asterisks). Fzo¹/fzo² mutant spermatids arrested at the mid-spermatid-elongation stage and did not complete nuclear condensation (C). In these mutants active-drICE (green) accumulated in the more advanced elongating spermatid cysts despite the immature nuclei (blue), demonstrating that drICE activation occured independently of nuclear differentiation [C, elongated spermatid cysts. Scale bars in (A) and (C) are 50 μm, and in (B) is 25 μm].

FIG. 6 demonstrates expression of Drosophila orthologues of the apoptosome complex during spermatogenesis. Dronc (green), a caspase-9 orthologue, accumulated along fully elongated spermatid cysts (arrowheads) and was depleted from individualized spermatozoa (arrow) (A). As the IC progressed caudally, Dronc expression increased within the CB (arrowhead) (B). The Drosophila orthologue of Apaf-1, hac-1 was expressed in spermatocytes (C). Transcription of hac-1 was visualized using the 1(2)k11502 line containing a P-lacZ insertion in the hac-1 promoter. In this line, lacZ expression (green) mimics the mRNA pattern of hac-1 (Zhou et al., 2000). Full-length inactive drICE (green) was expressed uniformly in pre-individualizing spermatid cysts (arrowheads), demonstrating that pro-drICE was expressed in early stages of spermatogenesis (D), however, drICE was activated by post-translation modification only after the assembly of the IC Scale bar, ?50 μm.

FIG. 7 demonstrates the specificity of the anti-Dronc polyclonal antibody, which detects a single band of the predicted size for the Dronc protein on Western blots (A). The two right lanes represent differences in loading of adult Drosophila protein extract. The anti-Dronc antibody stains ectopically expressed dominant negative (DN) Dronc in Drosophila wing imaginal discs (B). DN-Dronc was expressed in the posterior compartment of transgenic flies using engrailed-Gal4/UAS-pro-dronc-DN, and probed with antibody. The dominant negative form of Dronc was used since the natural form kills cells in which it is expressed.

FIG. 8A schematically depicts the genomic organization of the two cytochrome c genes and transposon insertions. The map illustrates the cyt-c-d and cyt-c-p exons (thick lines in black and grey), introns (thin lines), and insertion points in the 5′ UTR of cyt-c-d (exon-1) of the P-element transposons in strains EP(2)2305, EP(2)2049, bln¹, and the 1(2)k13905 allele, which bears a P-lac W insertion in the first nucleotide of cyt-c-p intron (triangles). The predicted ORF for cyt-c-d (ATG-TAG within exon-2) encodes a protein of 105 aa long. The cyt-c-p gene is located 241 bp downstream of cyt-c-d. The predicted ORF for cyt-c-p (ATG-TAA within exon-2) encodes a protein of 108 amino acids.

FIG. 8B demonstrates the cyt-c-d expression in WT and bln¹ mutants. Northern Blot analysis of adult male poly(A+) RNA of wild-type and bln¹ mutants probed for expression of the 3′ UTR region of cyt-c-d provided a single band of the predicted size for cyt-c-d (0.87 kb) in wild-type (wt, right lane), but not in the bln¹ mutant (left lane). The Drosophila rp49 gene served as the control for equal sample loading.

FIG. 9 demonstrates that functional cyt-c-d is involved in caspase activation at the onset of spermatid individualization. Active drICE is present in testes from wild-type (black arrows pointing at CBs and WBs), (A) but not bln¹ homozygous mutants (B). CM1 positive spermatids were readily detected in wild-type individualizing cysts (green, white arrow) (C), but not in bln¹ mutant testis (D) and (F), although bln¹ spermatids showed no other obvious morphological defects, including normal IC assembly (D, arrowheads) Hephaestus (heph²) mutants showed strong immunoreactivity with CM1 despite other severe defects in the individualization process (E), as did ten other mutants with defects in spermatid individualization (data not shown). Staining with mAB 2G8 (G), which detects cytochrome c only in apoptotic cells, stains the mitochondria of round spermatids (arrowheads) and then acquires a punctate expression pattern in elongated spermatids typical of mitochondrial protein expression at this stage (H) (Note that the giant mitochondrion of round spermatids can be easily visualized by DAPI due to staining of the mitochondrial DNA. Scale bars?? 50 μm).

FIG. 10 demonstrates that mutant phenotypes are a result of P-element insertion in the cyt-c-d gene. Panel A schematically depicts excision of the transposon in the bln¹ strain. Females homozygous for the bln¹ allele (2^{nd} chromosome) were crossed to Scutoid (Sco)/CyO males, which bear the transposase-producing Δ2-3 ‘jumpstart’ allele marked by kinked (ki) on the 3^{rd} chromosome. Progeny, which contain both a balanced bln¹ allele and the transposase were crossed to the original bln¹ strain. Groups of 5 males containing potential excisions (*) over the bln¹ allele were allowed to copulate with wild-type females for 4 days for a fertility test. Using this strategy, fertile males with high frequency were recovered and used to establish revertant stocks. Complete revertants for the two additional cyt-c-d P-element insertions were obtained (data not shown). Panel B demonstrates restoration of drICE-activation and individualization in bln¹ revertants. Testes of fertile males were dissected and analyzed by immunocytochemistry for the presence of active-drICE (green) and proper individualization (early CBs, the IC in red is marked by anti-lamin Dmo). Caspase activation and individualization was indistinguishable from wild type in these revertant males.

FIG. 11 demonstrates that dbruce is involved in protection of spermatid nuclei from apoptotic damage. dbruce −/− mutants have hypercondensed and degenerate nuclei (B) as compared to wild type (A). Mutant spermatid nuclei are significantly more condensed (arrowheads) and rounded (arrow and arrowheads) and are scattered throughout the cyst. In addition, there are fewer nuclei, and many nuclei are stained faintly (yellow arrows and enlargement in a subset), presumably because they undergo degeneration. Although an IC forms, it is highly reduced and scattered (red). Scale bars=20 μm.

FIG. 12 demonstrates the generation of sept4 null mutant mice. A schematic depiction of the targeting strategy for deletion of the murine sept4 locus is shown (A). Southern blot analysis indicated correct targeting of the sept14 locus.

FIG. 13 schematically depicts alternative transcripts of the sept4 locus.

FIG. 14 demonstrates that loss of septin 4 protein expression produces no effect on pre- or post-meotic stages of spermatogenesis in testes of sept4 null mutants (A, B), nor does the mutation result in qualitatively or quantitatively different mature sperm produced and stored in the epididymis as compared to wild-type (wild-type controls, 22×10⁶, versus mutants 20×10⁶ sperm) (C).

FIG. 15 demonstrates a marked difference in the spermatids produced in mice genetically disrupted for the sept4 locus. A schematic diagram of male reproductive tissue (A) and healthy sperm (B,C) serve as a comparison for the severe defects exhibited in sperm isolated from null mutant mice (D-G). Following passage through the Epididymis, bent sperm appear in the null mutants, as opposed to sperm isolated from controls.

FIG. 16 demonstrates the resulting absence of the annulus in sperm isolated from the null mutants. Electron microscopy of mutant sperm demonstrates the absence of an annulus (A, and shown in greater detail in B), which produces complete immobility in the mutant sperm as compared to controls (C, and D).

FIG. 17 demonstrates by transmission electron microscopy the resulting bent sperm in mutant mice, produced as a consequence of the absence of the sperm annulus.

FIG. 18 demonstrates by light microscopy a role for caspase 3 in late stages of mammalian spermatogenesis. Histologic sections of seminiferous tubules of fertile adult normal males demonstrated caspase 3 expression in cytoplasmic droplets of spermatids, by CM1 immunostaining (arrows, A, B).

FIG. 19 demonstrates by brightfield and fluorescent microscopy that caspase 3 activity is found in late stages of spermatid differentiation. Caspase 3 is localized to the cytoplasmic droplet and head region of differentiated spermatids (A-C) (A-brightfield, B-CM-1 staining; C-overlay of A & B). Mature spermatozoa express caspase 3 in cytoplasmic droplets and acrosomes (D-F) (D—brightfield, E-CM-1 staining; F—overlay of D & E).

FIG. 20 demonstrates by brightfield and fluorescent microscopy that unlike caspase 3 activity, caspase 9 activity is restricted to the principal piece of the sperm tail in epididymal cauda sperm (A). Similarly, caspase 6 activity is restricted to the principal piece of the sperm tail (C) (B-brightfield, C-caspase 6 specific antibody staining; D—overlay of B & C), which essentially mirrors that of caspase 9 activity.

### DETAILED DESCRIPTION OF THE INVENTION

This invention provides, in one embodiment, methods for the regulation of spermatid differentiation. This invention provides, in other embodiments, applications of regulated spermatid differentiation, including the treatment of infertility, contraception and pest control.

Spermatogenesis occurs in the seminiferous tubules of the testes of sexually mature male mammals. Spermatogonia undergo mitosis, giving rise to two daughter cells; one may remain near the basement membrane as a spermatogonium and the other may develop, through subsequent rounds of mitosis, into a primary spermatocyte. The primary spermatocyte then enters meiosis and gives rise to haploid spermatids. These spermatids undergo a metamorphosis, maturing into spermatozoa, which are then released into the lumen of the seminiferous tubule.

Caspase Involvement in Spermatogenesis:

Apoptotic proteins play an important role in spermatogenesis. Caspase activation is shown herein to be intimately associated with the individualization process during sperm cell terminal differentiation (Example 4, hereinbelow), and is an absolute requirement for spermatid individualization. Cystic bulges and waste bags, which contain cytoplasm expelled from differentiating spermatids, display many features of apoptotic corpses and contain all the components required for a functional apoptosome (Example 6).

As used herein, the term “apoptosis” is defined as a mechanism of programmed cell death, the most common form of physiological (as opposed to pathological) cell death. Apoptosis is an active process requiring metabolic activity by the dying cell; often characterized by shrinkage of the cell, cleavage of the DNA into fragments that give a so-called “laddering pattern” on gels and by condensation and margination of chromatin.

A key feature of apoptosis is the activation of caspases. Once activated, caspases mediate apoptosis by cleaving specific intracellular proteins, including proteins of the cytoskeleton and cytosol (Thornberry and Lazebnik, 1998; Cryns and Yuan, 1998). Caspase activation is a critical and necessary step in spermatid differentiation.

In one embodiment, the invention provides a method of stimulating or enhancing spermatid differentiation, comprising the step of contacting a spermatogenic cell with an agent that stimulates or enhances caspase expression, activity or function in the cell, thereby stimulating or enhancing spermatid differentiation.

The term “caspase”, as referred to herein, is an aspartic acid specific cysteine protease. Caspases may comprise isoforms of individual caspases, each isoform being encoded by the same gene. By way of example, α and β isoforms of caspase 8 have been identified, and are both encoded by the same gene. Discussion herein of a caspase is intended to refer to all isoforms of the caspase. For example, discussion of caspase 8 refers to the α and β isoforms of caspase 8.

Currently, there are at least 14 known caspase genes in mammals, named caspase-1 through caspase-14. Caspases are found in a myriad of organisms, including human, mouse, insect (e.g., Drosophila), and other invertebrates (e.g., C. elegans). Examples of caspases and their respective alternative names, as referred to in scientific literature are: Caspase-1 or ICE; Caspase-2 or ICH-1; Caspase-3 or CPP32, Yama, apopain; Caspase-4 or ICErel11; TX, ICH-2; Caspase-5 or ICErel111; TY; Caspase-6 or Mch2; Caspase-7 or Mch3, ICE-LAP3, CMH-1; Caspase-8 or FLICE; MACH; Mch5; Caspase-9 or ICE-LAP6; Mch6; and Caspase-10 or Mch4 or FLICE-2.

In one embodiment, the caspase whose expression, function or activity is modulated may correspond to or be homologous to a caspase such as that disclosed in NCBI's Entrez protein database, having the Accession number: AAP36898, AAP36827, AAP36282, AAP36279, AAP35904, AAP35557, AAP35329, NP476974, NP2035221, NP203520, NP203519, NP001219, NP203126, NP203125, NP203124, NP150636, NP150635, NPI 16787, NP001217, NP127463, NP001218, NP001214, NP116759, NP116758, Q92851, Q14790, O01382, AAM44398, AAH34262, P97864, P55210, X65019, U13021, U13737, U25804, U28015, U20536, U37448, U60520, U56390, U60519 or P42575.

In another embodiment, the caspase whose expression, function or activity is modulated may be encoded by a nucleotide sequence that corresponds to or is homologous to a sequence such as that disclosed in NCBI's Entrez nucleotide database, having the Accession number: NM033357, NM033356, NM033355, NM01228, NM0033340, NM001227, NM033294, NM033293, NM001223, NM032992, NM001226, NM032996, NM001229, NM004346, NM032991, NM032977, NM032974, NM001230, NM033358, NM033295, NM033292, NM031775, NM022260, NM012922, NM012114, NM009807, NM004347, BC034262, BC005428, BC008152, BC006757, AY219866, AY214168, BD085817, BD085816, BD085815, BD085814, BD085813, BD085812 or BD073936.

The term “homology”, as used herein, when in reference to any protein or peptide, indicates a percentage of amino acid residues in the candidate sequence that are identical with the residues of a corresponding native polypeptide, after aligning the sequences and introducing gaps, if necessary, to achieve the maximum percent homology, and not considering any conservative substitutions as part of the sequence identity. Neither N- or C-terminal extensions nor insertions shall be construed as reducing identity or homology. Methods and computer programs for the alignment are well known in the art.

The term “homology”, as used herein, when in reference to any nucleic acid sequence similarly indicates a percentage of nucleotides in a candidate sequence that are identical with the nucleotides of a corresponding native nucleic acid sequence.

Homology may be determined in the latter case by computer algorithm for sequence alignment, by methods well described in the art. For example, computer algorithm analysis of nucleic acid sequence homology may include the utilization of any number of software packages available, such as, for example, the BLAST, DOMAIN, BEAUTY (BLAST Enhanced Alignment Utility), GENPEPT and TREMBL packages.

An additional means of determining homology is via determination of candidate sequence hybridization, methods of which are well described in the art (See, for example, “Nucleic Acid Hybridization” Hames, B. D., and Higgins S. J., Eds. (1985); Sambrook et al., 1989, Molecular Cloning, A Laboratory Manual, (Volumes 1-3) Cold Spring Harbor Press, N.Y.; and Ausubel et al., 1989, Current Protocols in Molecular Biology, Green Publishing Associates and Wiley Interscience, N.Y). For example methods of hybridization may be carried out under moderate to stringent conditions, to the complement of a DNA encoding a native caspase peptide. Hybridization conditions being, for example, overnight incubation at 42° C. in a solution comprising: 10-20% formamide, 5×SSC (150 mM NaCl, 15 mM trisodium citrate), 50 mM sodium phosphate (pH 7.6), 5× Denhardt's solution, 10% dextran sulfate, and 20 μg/ml denatured, sheared salmon sperm DNA.

Protein and/or peptide homology for any amino acid sequence listed herein may be determined by methods well described in the art, including immunoblot analysis, or via computer algorithm analysis of amino acid sequences, utilizing any of a number of software packages available, via established methods. Some of these packages may include the FASTA, BLAST, MPsrch or Scanps packages, and may employ the use of the Smith and Waterman algorithms, and/or global/local or BLOCKS alignments for analysis, for example.

According to this aspect of the invention, the caspases may include wild-type protein sequences, as well as other variants (including alleles) of the native protein sequence. Briefly, such variants may result from natural polymorphisms or may be synthesized by recombinant methodology, and differ from wild-type protein by one or more amino acid substitutions, insertions, deletions, or the like. As will be appreciated by those skilled in the art, a nucleotide sequence encoding a caspase or variant may differ from the known native sequences, due to codon degeneracies, nucleotide polymorphisms, or amino acid differences. Each of these represents an additional embodiment of the invention.

As used herein, the terms “homology”, “homologue” or “homologous”, in any instance, indicate that the sequence referred to, whether an amino acid sequence, or a nucleic acid sequence, exhibits, in one embodiment at least 70% correspondence with the indicated sequence. In another embodiment, the amino acid sequence or nucleic acid sequence exhibits at least 72% correspondence with the indicated sequence. In another embodiment, the amino acid sequence or nucleic acid sequence exhibits at least 75% correspondence with the indicated sequence. In another embodiment, the amino acid sequence or nucleic acid sequence exhibits at least 77% correspondence with the indicated sequence. In another embodiment, the amino acid sequence or nucleic acid sequence exhibits at least 80% correspondence with the indicated sequence. In another embodiment, the amino acid sequence or nucleic acid sequence exhibits at least 82% correspondence with the indicated sequence. In another embodiment, the amino acid sequence or nucleic acid sequence exhibits at least 85% correspondence with the indicated sequence. In another embodiment, the amino acid sequence or nucleic acid sequence exhibits at least 87% correspondence with the indicated sequence. In another embodiment, the amino acid sequence or nucleic acid sequence exhibits at least 90% correspondence with the indicated sequence. In another embodiment, the amino acid sequence or nucleic acid sequence exhibits at least 92% correspondence with the indicated sequence. In another embodiment, the amino acid sequence or nucleic acid sequence exhibits at least 95% or more correspondence with the indicated sequence. In another embodiment, the amino acid sequence or nucleic acid sequence exhibits 95%-100% correspondence to the indicated sequence. Similarly, as used herein, the reference to a correspondence to a particular sequence includes both direct correspondence, as well as homology to that sequence as herein defined.

The terms “caspase modulator”, “modulates/ing caspase activity” or “modulates/ing caspase function” are all to be considered synonymous, and refer to a nucleic acid, peptide, compound or composition, which stimulates, increases, abrogates or decreases caspase expression, activity or function. Caspase modulators include caspase activators, wherein the expression and/or activity of a caspase is greater, as a result of the presence of the compound. It is to be understood that any molecule that results in stimulated or increased caspase expression and/or activity, or whereby caspase function is stimulated and/or increased is to be considered as part of this invention. Caspase modulators include caspase inhibitors, as well, wherein the expression and/or activity of a caspase is less, as a result of the inhibitor compound. Any molecule which produces a decrease or inhibition or abrogation in caspase expression and/or activity and/or function is to be considered as part of this invention as well, and is considered in the context of caspase modulators.

Modulators of the present invention may be isolated or procured from a variety of sources, such as bacteria, fungi, plants, parasites, libraries of chemicals, peptides or peptide derivatives, antibodies, and the like. Modulators may also be rationally designed, based on the protein structures determined from X-ray crystallography.

Stimulating or Enhancing Caspase Expression, Activity or Function:

Stimulation or increasing caspase expression, activity or function can be accomplished through a variety of means well known in the art.

In one embodiment, stimulation or increasing caspase expression, activity or function is accomplished via the provision of a caspase to an affected cell. Purified or recombinant protein may be supplied, thus providing for increased caspase expression, activity or function.

In one embodiment, a caspase of choice can be purified from bulk cell culture, by methods well described in the art (see, for example, “Cell Biology: A Laboratory Handbook”, Volumes I-III Cellis, J. E., ed (1994) & “Strategies for Protein Purification and Characterization—A Laboratory Course Manual” CSHL Press (1996)). In another embodiment, an active fragment or peptide derivative of the caspase protein is prepared, and serves to modulate caspase expression, activity or function, by methods well described in the art.

In one embodiment, the peptides include, but are not limited to, fragments of native polypeptides from any animal species, including degradation products, synthetically synthesized peptides or recombinant peptides, variants and derivatives of native polypeptides and their fragments, provided that they have a biological activity in common with a respective native polypeptide. “Fragments” comprise regions within the sequence of a mature native polypeptide. The term “derivative” is meant to include amino acid sequence and glycosylation variants, and covalent modifications of a native polypeptide, whereas the term “variant” refers to amino acid sequence and glycosylation variants within this definition. In another embodiment, the agent of the invention may comprise a peptidomimetic (typically, synthetically synthesized peptides), such as peptoids and semipeptoids which are peptide analogs, which may have, for example, modifications rendering the peptides more stable while in a body or more capable of penetrating into cells. Such modifications include, but are not limited to N terminal, C terminal or peptide bond modification, including, but not limited to, backbone modifications, and residue modification, each of which represents an additional embodiment of the invention. Methods for preparing peptidomimetic compounds are well known in the art and are specified, for example, in Quantitative Drug Design, C. A. Ramsden Gd., Chapter 17.2, F. Choplin Pergamon Press (1992).

It is to be understood that any peptide of the present invention may be isolated, generated synthetically, obtained via translation of sequences subjected to any mutagenesis technique, as well as obtained via protein evolution techniques, well known to those skilled in the art.

In another embodiment, recombinant protein production is a means whereby caspases and other agents/proteins of choice of the invention are produced. The recombinant proteins may then be introduced into an organism, or directly into cells of an organism of choice. Methods of generating recombinant caspases are further described hereinbelow.

Caspase protein expression can be verified by methods including, but not limited to, HPLC, mass spectroscopy, GLC, immunohistochemistry, ELISA, RIA, or western blot analysis. When using a method, which relies on the immunological properties of the protein in question, antibodies against the entire protein or a peptide derived from the protein can be raised and used. Alternatively, and according to another embodiment of the present invention, an expressed sequence tag (EST) encoding a known tag peptide sequence (for example HIS tag) can be inserted into the recombinant protein either on the 5′ or the 3′ end thus the HIS-tag proteins can be isolated using His-Tag Ni-column chromatography. Similarly, in still another preferred embodiment of the present invention, a polycistronic recombinant nucleic acid including an Internal Ribosome Entry Site (IRES) sequence residing between the sequence encoding the protein of interest and a sequence encoding a reporter protein may be generated, so as to enable detection of a known marker protein. Additional marker proteins may be incorporated, or comprise the recombinant proteins, and as such encompass still further preferred embodiments of the present invention.

Caspases may be provided, according to another embodiment, via introduction of an expression vector comprising a nucleic acid sequence encoding for a caspase.

By “vector” what is meant is a nucleic acid construct containing a sequence of interest that has been subcloned within the vector. In one embodiment, the nucleic acid sequence encodes for a caspase or a functional fragment thereof.

To generate the vectors of the present invention, the polynucleotide segments encoding caspases and other sequences of interest can be ligated into commercially available expression vector systems suitable for transducing/transforming eukaryotic or prokaryotic cells and for directing the expression of recombinant products within the transduced/transformed cells. It will be appreciated that such commercially available vector systems can easily be modified via commonly used recombinant techniques in order to replace, duplicate or mutate existing promoter or enhancer sequences and/or introduce any additional polynucleotide sequences such as for example, sequences encoding additional selection markers or sequences encoding reporter genes.

According to another embodiment, nucleic acid vectors comprising the isolated nucleic acid sequences encoding for the protein of interest include a promoter for regulating expression of the isolated nucleic acid. Such promoters are known to be cis-acting sequence elements required for transcription as they serve to bind DNA dependent RNA polymerase, which transcribes sequences present downstream thereof.

A vector according to the present invention, may, in another embodiment further include an appropriate selectable marker. The vector may further include an origin of replication, and may be a shuttle vector, which can propagate both in prokaryotic, and in eukaryotic cells, or the vector may be constructed to facilitate its integration within the genome of an organism of choice. The vector according to this aspect of the present invention can be, for example, a plasmid, a bacmid, a phagemid, a cosmid, a phage, a virus or an artificial chromosome.

The caspase vector may, in another embodiment, comprise an isolated nucleic acid encoding a self-activating form of the caspase and a promoter operably linked to the isolated nucleic acid. Alternately, if the cell has been provided with one or more proteolytic enzymes capable of activating the zymogen form of the caspase, then the isolated nucleic acid may encode a non-self-activating form of the caspase. The promoter of the caspase vector may be an inducible promoter, or one that expresses the sequence constitutively.

Nucleic acid sequences encoding for modulators may be utilized as described, in the present invention. As used herein, the term “nucleic acid” refers to polynucleotide or to oligonucleotides such as deoxyribonucleic acid (DNA), and, where appropriate, ribonucleic acid (RNA) or mimetic thereof. The term should also be understood to include, as equivalents, analogs of either RNA or DNA made from nucleotide analogs, and, as applicable to the embodiment being described, single (sense or antisense) and double-stranded polynucleotides. This term includes oligonucleotides composed of naturally occurring nucleobases, sugars and covalent internucleoside (backbone) linkages as well as oligonucleotides having non-naturally-occurring portions, which function similarly. Such modified or substituted oligonucleotides are often preferred over native forms because of desirable properties such as, for example, enhanced cellular uptake, enhanced affinity for nucleic acid target and increased stability in the presence of nucleases.

As will be appreciated by one skilled in the art, a fragment or derivative of a nucleic acid sequence or gene that encodes for a protein or peptide can still function in the same manner as the entire, wild type gene or sequence. Likewise, forms of nucleic acid sequences can have variations as compared to wild type sequences, nevertheless encoding the protein or peptide of interest, or fragments thereof, retaining wild type function exhibiting the same biological effect, despite these variations. Each of these represents a separate embodiment of this present invention.

The nucleic acids can be produced by any synthetic or recombinant process such as is well known in the art. Nucleic acids can further be modified to alter biophysical or biological properties by means of techniques known in the art. For example, the nucleic acid can be modified to increase its stability against nucleases (e.g., “end-capping”), or to modify its lipophilicity, solubility, or binding affinity to complementary sequences.

DNA according to the invention can also be chemically synthesized by methods known in the art. For example, the DNA can be synthesized chemically from the four nucleotides in whole or in part by methods known in the art. Such methods include those described in Caruthers (1985). DNA can also be synthesized by preparing overlapping double-stranded oligonucleotides, filling in the gaps, and ligating the ends together (see, generally, Sambrook et al. (1989) and Glover et al. (1995)). DNA expressing functional homologues of the protein can be prepared from wild-type DNA by site-directed mutagenesis (see, for example, Zoller et al. (1982); Zoller (1983); and Zoller (1984); McPherson (1991)). The DNA obtained can be amplified by methods known in the art. One suitable method is the polymerase chain reaction (PCR) method described in Saiki et al. (1988), Mullis et al., U.S. Pat. No. 4,683,195, and Sambrook et al. (1989).

Methods for modifying nucleic acids to achieve specific purposes are disclosed in the art, for example, in Sambrook et al. (1989). Moreover, the nucleic acid sequences of the invention can include one or more portions of nucleotide sequence that are non-coding for the protein of interest. Variations in the DNA sequences, which are caused by point mutations or by induced modifications (including insertion, deletion, and substitution) to enhance the activity, half-life or production of the polypeptides encoded thereby, are also encompassed in the invention.

Caspase expression may be stimulated or enhanced in a cell by providing an expressible vector encoding the caspase to the cell and thereafter expressing the caspase. Alternatively, the caspase may be provided to the cell by integrating an isolated nucleic acid encoding the caspase into the genome of the cell and thereafter expressing the caspase.

In another embodiment, the agent that stimulates or enhances caspase expression, activity or function is a chemical compound, and may be a substituted indole-2-carboxylic acid benzylidene-hydrazide or analog thereof (see US 20020128292A1), or a benzodiazepine (see US 20010016583A1).

In one embodiment, the caspase modulators of the present invention may be supplied in native form. In another embodiment, a composition comprising the modulators is envisioned.

The effective dose and method of administration of a particular composition formulation comprising a caspase modulator can vary based on the particular application. Dosage may vary as a function of the type of caspase modulator employed and the route of administration.

Routes of administration of the nucleic acids, vectors, peptides, compounds and compositions of the invention include, but are not limited to oral or local administration, such as by aerosol, intramuscularly or transdermally, and parenteral application. Compositions can be administered in a variety of unit dosage forms depending upon the method of administration. Suitable unit dosage forms, include, but are not limited to powders, tablets, pills, capsules, lozenges, suppositories, etc. Transdermal administration may be accomplished by application of a cream, rinse, gel, etc. capable of allowing the active compounds to penetrate the skin. Parenteral routes of administration may include, but are not limited to, electrical or direct injection such as direct injection into a central venous line, intravenous, intramuscular, intraperitoneal, intradermal, or subcutaneous injection.

Compositions of the invention may include, but are not limited to: suspensions, oils, creams, and ointments applied directly to the skin or incorporated into a protective carrier such as a transdermal device (“transdermal patch”). Examples of suitable creams, ointments, etc. can be found, for instance, in the Physician's Desk Reference. Examples of suitable transdermal devices are described, for instance, in U.S. Pat. No. 4,818,540 issued Apr. 4, 1989 to Chinen, et al.

Compositions of this invention suitable for parenteral administration include, but are not limited to, sterile isotonic solutions. Such solutions include, but are not limited to, saline and phosphate buffered saline for injection into a central venous line, intravenous, intramuscular, intraperitoneal, intradermal, or subcutaneous injection.

Caspase Activation and Fertility:

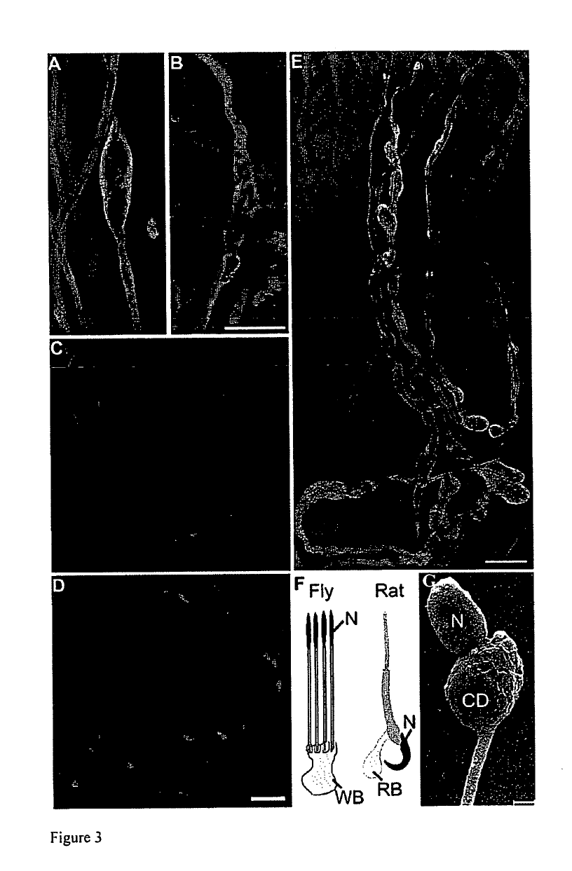

Caspase activation was shown to be an absolute requirement for spermatid differentiation. Administration of a caspase inhibitor resulted in poor cytoplasmic accumulation within CBs, resulting in arrest of the maturation pathway (FIG. 3B). Morphological evaluation of caspase inhibitor treated spermatids (FIG. 3E) demonstrated a striking resemblance to one of the most commonly seen abnormalities of human spermatozoa, known as cytoplasmic droplet sperm (FIG. 3F, right panel) (Hollanders and Carver-Ward J. A., 1996).

In one embodiment, there is provided a method of treatment of infertility in a male subject in need thereof, comprising contacting a spermatogenic cell in said subject with an agent that stimulates or enhances caspase expression or activity in said cell.

In order to treat infertility in a male, spermatogenic cells must be contacted with an agent stimulating or enhancing caspase expression, activity or function. In one embodiment, the spermatogenic cell is a spermatogonium. In another embodiment, the spermatogenic cell is a spermatocyte. In another embodiment, the spermatogenic cell is a spermatid.

As used herein, the term “contacting”, “contact” or “contacted” when in reference to a cell indicates both direct and indirect exposure of the cell to a nucleic acid, peptide, protein, vector, compound or composition of the invention. In one embodiment, contacting a cell may comprise direct injection of the cell through any means well known in the art, such as microinjection. It is also envisaged, in another embodiment, that supply to the cell is indirect, such as via provision in a culture medium that surrounds the cell, or via parenteral administration in a body of a subject in need, whereby the agent ultimately contacts a spermatogenic cell via peripheral circulation.

Protocols for introducing a nucleic acid or vector of the invention into cells may comprise, for example: direct DNA uptake techniques, virus, plasmid, linear DNA or liposome mediated transduction, or transfection, direct injection, magnetoporation, receptor-mediated uptake and others (for further detail see, for example, “Methods in Enzymology” Vol. 1-317, Academic Press, Current Protocols in Molecular Biology, Ausubel F. M. et al. (eds.) Greene Publishing Associates, (1989) and in Molecular Cloning: A Laboratory Manual, 2nd Edition, Sambrook et al. Cold Spring Harbor Laboratory Press, (1989), or other standard laboratory manuals). It is to be understood that any direct means or indirect means of intracellular access of a nucleic acid or vector of the invention is contemplated herein, and represents an embodiment thereof.

Cyt-c-d and Homlogues Thereof and Caspase Activation:

Mitochondria release several pro-apoptotic proteins into the cytosol in response to apoptotic stimuli (Green and Reed, 1998; Meier et al., 2000a). The best-studied case is the release of cytochrome c, which binds to and activates Apaf-1, which in turn leads to the activation of caspase-9 (Wang, 2001). A unique finding of the present invention is the involvement of a mitochondrially-located protein in spermatid differentiation. A specific form of cytochrome c, encoded by the cyt-c-d gene, was involved in caspase activation at the onset of spermatid individualization. Cyt-c-d loss-of-function mutants were viable, but male-sterile (Example 7). The mutants were defective in caspase activation and spermatid bulk cytoplasm extrusion, with a phenotype indistinguishable from spermatids treated with caspase inhibitors. Thus, according to another aspect of this invention, spermatid differentiation is a product of cyt-c-d signal transduction, resulting in caspase activation, in developing spermatids.

Sept4/ARTS Protein and Fertility:

Sept4/ARTS is a protein belonging to the septin family, which has been shown to positively regulate caspase3 activity (Larisch, Lotan et al, Nat Cell Biol. 2000 December; 2(12):915-21). A unique finding of the present invention is the involvement of the Sept4/ARTS protein in spermatid differentiation. Homozygous sept4/ARTS knockout mice were viable, but male-sterile. The sperm of the mutants exhibited a cytoplasmic droplet morphology, similar to a commonly seen abnormality of human spermatozoa (Example 4). Further, mice genetically disrupted for the sept4 locus were deficient in annulus formation, resulting in more than 90% of spermatids with bent morphology. Thus, according to another aspect of this invention, spermatid differentiation is affected by sept4 protein expression as well as sept4/ARTS signal transduction, resulting in caspase activation, in developing spermatids.

Dbruce Protein and Fertility:

Effector caspases, such as drICE, Dcp-1 and caspase-3, which have been shown to participate in spermatid maturation, also cleave a variety of nuclear targets, including lamins, I-CAD, and PARP, which is potentially damaging to the sperm nucleus. For spermatid maturation, the sperm nucleus must be protected against their lethal activity. One candidate for such a protective function is dbruce, the Drosophila orthologue of mammalian Bruce/Apollon. Bruce/Apollon proteins are E2 ubiquitin conjugating enzymes that are thought to inhibit apoptosis. dBruce contains a BIR domain, a motif found in IAPs, which may mediate its binding to caspases or Reaper/Hid/Grim-like (RHG) proteins, though the data does not support RHG-proteins as direct targets for dBruce. A unique finding of the present invention is the involvement of dBruce in spermatid differentiation. Screens for genetic modifiers of Reaper-induced apoptosis identified 13 alleles of the Drosophila IAP-related gene, dBruce (Agapite and Steller, unpublished results), among which alleles were homozygous viable but male-sterile, behaving as loss-of-function alleles. Moreover, examination of testes from the homozygous Bruce mutant mice exhibited abnormal sperm spermatogenesis and lack of functional sperm (Example 10). Thus, according to another aspect of this invention, spermatid differentiation is a product of dBruce activity, conferring protection from caspase activation, in developing spermatids.

In one embodiment, there is provided a method of stimulating or enhancing spermatid differentiation, comprising the step of contacting a spermatogenic cell with an agent that stimulates or enhances expression or activity of cyt-c-d, Sept4/ARTS, or Bruce in the cell, thereby stimulating or enhancing spermatid differentiation.

As used herein, the term “agent” refers to any molecule that performs the indicated function. In one embodiment, the agent is a nucleic acid sequence. In another embodiment, the agent is an expression vector comprising an indicated nucleic acid. In another embodiment, the agent is a peptide, or protein. In another embodiment, the agent is a small molecule. In another embodiment, the agent is derived or isolated from natural sources. In another embodiment, the agent is synthesized. It is to be understood that agent is meant to encompass any of these embodiments, and is meant to include any mediator of an indicated function.

In one embodiment, the cyt-c-d whose expression, function or activity is modulated may correspond to or be homologous to a cyt-c-d such as that disclosed in NCBI's Entrez protein database, having the Accession number: NP477164, P00034, XP215748, AAF53553, AAF86874 or CAA25901.

In another embodiment, the cyt-c-d whose expression, function or activity is modulated may be encoded by a nucleotide sequence that corresponds to or is homologous to a sequence such as that disclosed in NCBI's Entrez nucleotide database, having the Accession number: AC093097, AE00365, AQ073476, AQ074057, AW944049, X01761, NM057816, XM215748, NG000525 or AF210938.

In one embodiment, the Sept4/ARTS protein whose expression, function or activity is modulated may correspond to or be homologous to a Sept4/ARTS protein such as that disclosed in NCBI's Entrez protein database, having the Accession number: NP004040, P28661, NP004565, NPO35259, NP536340, NP536341, NP536342, O43236.

In another embodiment, the sept4/ARTS protein whose expression, function or activity is modulated may be encoded by a nucleotide sequence that corresponds to or is homologous to a sequence such as that disclosed in NCBI's Entrez nucleotide database, having the Accession number: AF176379, NT039520, NM011129, NM080415, NM080416, NM004574, NM080417.

In one embodiment, the Bruce protein whose expression, function or activity is modulated may correspond to or be homologous to a Bruce protein such as that disclosed in NCBI's EntTrez protein database, having the Accession number: CAA76720, NPO31592, NP850218, NP179284, NPO57336, AA073060, Q9NR09, AAF75772.

In another embodiment, the Bruce protein whose expression, function or activity is modulated may be encoded by a nucleotide sequence that corresponds to or is homologous to a sequence such as that disclosed in NCBI's Entrez nucleotide database, having the Accession number: NM007566, CB552739, CB551841, NM179887, NM127245, NM016252 AY221595, AY221594, AK091644, AF265555.

The spermatogenic cell, as defined hereinabove, is contacted with an agent that stimulates or enhances expression, activity or function of cyt-c-d or Sept4/ARTS in the cell. In one embodiment, stimulated or enhanced cyt-c-d or ARTS expression, activity or function results in stimulated or enhanced caspase expression, activity or function.

Treatments for Infertility:

In another embodiment, the invention provides a method of treatment of infertility in a male subject in need thereof, comprising contacting a spermatogenic cell in the subject with an agent that stimulates or enhances expression, activity or function of cyt-c-d or Sept4/ARTS in the cell.

It is to be understood that contacting the cell with any agent of the invention is as herein defined, and may be effected, in one embodiment, in vivo or, in another embodiment, in vitro. In another embodiment, spermatogenic cells are ex-vivo contacted with an agent of the invention, and may be further utilized, for example in vitro fertilization procedures, or other manipulations. The spermatogenic cell may therefore be returned to the subject from whom the cells were obtained, or in another embodiment, may be utilized for insemination.

According to this aspect of the invention, in one embodiment, the spermatogenic cells are introduced into the individual tubules of the subject from whom the cells were obtained. For example, the subject can be anesthetized and the testis (or testes) surgically exposed. By micromanipulation methods a thin glass needle can be introduced into exposed tubules, with each tubule injected with a solution containing the spermatogenic cells. Alternatively, the spermatogenic cells can be injected into other parts of the tubular system e.g. the lumen of the rete testes. Injections may utilize either fine stainless-steel needles or fine pulled-glass capillaries loaded with spermatogenic cells. A micromanipulator is used to direct the tip of such an instrument to penetrate the rete testis or the seminiferous tubule. The cells are expelled and will back-fill the seminiferous tubules.

In another embodiment, the spermatogenic cells that have been contacted with an agent of the invention are subsequently utilized in assisted reproductive technologies (ARTs) (i.e. a procedure for contacting a sperm with an ovum to initiate a pregnancy). Examples of ARTs include in vitro fertilization (IVF), gamete intrafallopian transfer (GIFT) intrauterine insemination (IUI) and intracytoplasmic sperm injection (ICSI). Procedures for performing ARTs are well-known to practitioners, and it is expected that additional ARTs will be developed over time.

According to another aspect of the invention, spermatogenic cells are contacted with a growth factor or hormone and an agent of the present invention. In one embodiment, the growth factor may be an insulin-like growth factor (IGF) I or II or an insulin growth factor binding protein (IGFBP).

In another embodiment, the hormone may be growth hormone, or a gonadotropin, including follicle stimulating hormone (FSH), luteinizing hormone (LH), luteinizing hormone releasing hormone (LH-RH) or an androgen, such as testosterone or androstenedione, or a variant thereof.

Animal models for studying infertility can be designed, in another embodiment of the invention, based upon cyt-c-d, sept4/ARTS, or Bruce involvement in caspase-mediated spermatid differentiation. Animals genetically disrupted for the cyt-c-d or Bruce gene can be engineered by methods well described in the art (see, for example U.S. Pat. Nos. 4,736,866 and 4,870,009 and Hogan, B. et al., (1986) A Laboratory Manual, Cold Spring Harbor, N.Y., Cold Spring Harbor Laboratory). Alternatively, mutants can be generated, such as those disclosed hereinbelow, producing cyt-c-d or Bruce protein with varying function/activity levels. In another embodiment, animals disrupted for the sept4/ARTS gene, such as those disclosed hereinbelow, can be generated. In another embodiment, mutants can be generated producing sept4/ARTS protein with varying function/activity levels. These animals can, in one embodiment, be utilized to determine downstream effects of the cyt-c-d, sept4/ARTS, or Bruce proteins function on spermatid maturation. In another embodiment, such animals can be utilized to assess the efficacy and toxicity, etc. of variants of these proteins, or in another embodiment, their ability to stimulate, enhance, or protect from caspase expression, activity or function. Animals genetically disrupted for a caspase gene may similarly be generated, or available strains may be utilized, and such animals may similarly provide information as to the efficacy, toxicity, therapeutic potential of the agents of the present invention, in terms of the latter's ability to stimulate spermatid differentiation. In another embodiment, the animals disclosed herein may serve as a model for the study of impaired spermatid differentiation or infertility.

In another embodiment, there is provided a method of identifying an agent that stimulates or increases the fertility rate of a male mammal comprising administering a candidate agent to a non-human male transgenic animal genetically disrupted for caspase, cyt-c-d, sept4/ARTS, or Bruce gene function and determining the effect of the candidate agent on the fertility rate of the non-human male transgenic animal, wherein an increased fertility rate indicates that the candidate biologically active agent increases the fertility rate of a mammal.

As used herein, the term “candidate agent” refers to an agent, as defined hereinabove, that is being considered for its ability to generate a desired outcome, in the context of the indicated parameter being measured. A candidate agent may demonstrate, in one embodiment, a therapeutic effect directly, such as for example, in directly enhancing caspase activity resulting in spermatid differentiation. In another embodiment, a candidate agent may demonstrate a therapeutic effect indirectly, such as for example, in enhancing expression or activity of cyt-c-d, Sept4/ARTS, or Bruce, which ultimately results in spermatid differentiation.

Inhibiting Caspase-Mediated Spermatid Differentiation:

In another embodiment, there is provided a method of inhibiting or abrogating spermatid differentiation, comprising the step of contacting a spermatogenic cell with an agent that inhibits or abrogates caspase expression or activity in said cell, thereby inhibiting or abrogating spermatid differentiation.

According to this aspect of the invention, the agent that inhibits caspase expression, activity or function may comprise any embodiment described hereinabove. In one embodiment, the agent is the compound Z-VAD (Fraser and Evan, 1997), Cbz-Pro-Asp-fmk (see WO 91/15557), Cbz-Thz-Asp-fmk (see WO 99/477154) or 4-ClCbz-Val-Asp-fmk (see WO 00/61542), or any chemical moiety that is effective in inhibiting caspase expression, activity or function.

In another embodiment, the agent that inhibits caspase expression, activity or function comprises a nucleic acid. The nucleic acid may, in one embodiment, be DNA, or in another embodiment, the nucleic acid is RNA. In other embodiments, the nucleic acid may be single or double stranded.

In another embodiment, the agent is a nucleic acid that is antisense in orientation to a sequence encoding for a caspase.

According to this aspect of the invention, inhibition of caspase expression, activity or function is effected via the use of antisense oligonucleotides, which are chimeric molecules, containing two or more chemically distinct regions, each made up of at least one nucleotide. These chimeric oligonucleotides typically contain at least one region wherein the oligonucleotide is modified so as to confer upon the oligonucleotide an increased resistance to nuclease degradation, increased cellular uptake, and/or increased binding affinity for the target polynucleotide. An additional region of the oligonucleotide may serve as a substrate for enzymes capable of cleaving RNA:DNA or RNA:RNA hybrids, which according to this aspect of the invention, serves as a means of gene silencing via degradation of specific sequences. Cleavage of the RNA target can be routinely detected by gel electrophoresis and, if necessary, associated nucleic acid hybridization techniques known in the art.

The chimeric antisense oligonucleotides may, in one embodiment, be formed as composite structures of two or more oligonucleotides and/or modified oligonucleotides, as is well described in the art (see, for example, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922), and can, in another embodiment, comprise a ribozyme sequence.

Inhibition of caspase expression, activity or function is effected, in another embodiment, via the use of small interfering RNAs, which provides sequence-specific inhibition of gene expression. Administration of double stranded/duplex RNA (dsRNA) corresponding to a single gene in an organism can silence expression of the specific gene by rapid degradation of the mRNA in affected cells. This process is referred to as gene silencing, with the dsRNA functioning as a specific RNA inhibitor (RNAi). RNAi may be derived from natural sources, such as in endogenous virus and transposon activity, or it can be artificially introduced into cells (Elbashir S M, et al (2001). Nature 411:494-498) via microinjection (Fire et al. (1998) Nature 391: 806-11), or by transformation with gene constructs generating complementary RNAs or fold-back RNA, or by other vectors (Waterhouse, P. M., et al. (1998). Proc. Natl. Acad. Sci. USA 95, 13959-13964 and Wang, Z., et al. (2000). J. Biol. Chem. 275, 40174-40179). The RNAi mediating mRNA degradation, in one embodiment, comprises duplex or double-stranded RNA, or, in other embodiments, include single-stranded RNA, isolated RNA (partially purified RNA, essentially pure RNA, synthetic RNA, recombinantly produced RNA), as well as altered RNA that differs from naturally occurring RNA by the addition, deletion and/or alteration of one or more nucleotides.

When referring to nucleic acid sequences utilized as modulators in this invention, it is to be understood that such reference allows for the incorporation of non-nucleotide material, which may be added, for example, to the end(s) of the nucleotide sequence, including for example, terminal 3′ hydroxyl groups, or internal additions, at one or more nucleotides. Nucleic acids may, in another embodiment, incorporate non-standard nucleotides, including non-naturally-occurring nucleotides. Alterations may also include the construction of blunt and/or overhanging ends. Collectively all such altered nucleic acids may be referred to as analogs, and represent contemplated embodiments of the invention.

In another embodiment, caspase expression can be inhibited/downregulated simply by “knocking out” the gene. Typically this is accomplished by disrupting the caspase gene, the promoter regulating the gene or sequences between the promoter and the gene. Such disruption can be specifically directed to a caspase by homologous recombination where a “knockout construct” contains flanking sequences complementary to the domain to which the construct is targeted. Insertion of the knockout construct (e.g. into the caspase gene) results in disruption of that gene. The phrases “disruption of the gene” and “gene disruption” refer to insertion of a nucleic acid sequence into one region of the native DNA sequence (usually one or more exons) and/or the promoter region of a gene so as to decrease or prevent expression of that gene in the cell as compared to the wild-type or naturally occurring sequence of the gene.

Knockout constructs can be produced by standard methods known to those of skill in the art. The knockout construct can be chemically synthesized or assembled, e.g., using recombinant DNA methods. The DNA sequence to be used in producing the knockout construct is digested with a particular restriction enzyme selected to cut at a location(s) such that a new DNA sequence encoding a marker gene can be inserted in the proper position within this DNA sequence. The proper position for marker gene insertion is that which will serve to prevent expression of the native gene; this position will depend on various factors such as the restriction sites in the sequence to be cut, and whether an exon sequence or a promoter sequence, or both is (are) to be interrupted (i.e., the precise location of insertion necessary to inhibit promoter function or to inhibit synthesis of the native exon).

It is to be understood that the above nucleic acids may be delivered to spermatogenic cells in one embodiment, in their native form, or, in another embodiment within an expression vector that is competent to transfect cells in vitro and/or in vivo.

Methods for introducing nucleic acids into cells in vivo, ex vivo and in vitro are well known to those skilled in the art, some of which have been described hereinabove. Additional embodiments include the use of lipid or liposome based delivery (WO 96/18372; WO 93/24640; Mannino and Gould-Fogerite (1988) BioTechniques 6(7): 682-691; Rose U.S. Pat. No. 5,279,833; WO 91/06309; and Feigner et al. (1987) Proc. Natl. Acad. Sci. USA 84: 7413-7414) or the use of replication-defective retroviral vectors comprising a nucleic acid sequence as described (see, e.g., Miller et al. (1990) Mol. Cell. Biol. 10:4239 (1990); Kolberg (1992) J. NIH Res. 4: 43, and Cometta et al. (1991) Hum. Gene Ther. 2: 215) [For additional methods of delivery see: Anderson, Science (1992) 256: 808-813; Nabel and Feigner (1993) TIBTECH 11: 211-217; Mitani and Caskey (1993) TIBTECH 11: 162-166; Mulligan (1993) Science, 926-932; Dillon (1993) TIBTECH 11: 167-175; Miller (1992) Nature 357: 455-460; Van Brunt (1988) Biotechnology 6(10): 11491154; Vigne (1995) Restorative Neurology and Neuroscience 8: 35-36; Kremer and Perricaudet (1995) British Medical Bulletin 51(1) 31-44; Haddada et al. (1995) in Current Topics in Microbiology and Immunology, Doerfler and Bohm (eds) Springer-Verlag, Heidelberg Germany; and Yu et al., (1994) Gene Therapy, 1: 13-26].

In another embodiment, caspase expression, activity or function is inhibited or abrogated via the expression of an inhibitor in a desired cell. In one embodiment, expression of the baculoviral broad-spectrum caspase inhibitor p35 (Bump et al., 1995; Hay et al., 1994, and Example 3) inhibits or abrogates caspase expression, activity or function, thereby inhibiting spermatid differentiation.

Spermatid differentiation may be determined via a variety of methods well known in the art. In one embodiment, inhibition of spermatid differentiation may be determined morphologically. Formation of RBs, CBs and WBs as described herein, can readily be observed microscopically. In another embodiment, spermatid differentiation may be assessed via sample uptake of acridine orange, whereby dye incorporation highlights the formation of RBs, CBs and WBs. In another embodiment, spermatid differentiation may be assessed via surface markers unique to post-differentiation spermatids such as protamines 1 and 2 and transition protein 2 (Fujii et al, EMBO Reports 3: 367-372).

Cyt-c-d Inhibition:

Caspase activated spermatid differentiation was demonstrated herein to involve cyt-c-d, Sept4/ARTS, and Bruce (Examples 7 & 8), as described. Inhibition of cyt-c-d or Sept4/ARTS expression, activity or function is, in another embodiment, a means of inhibiting caspases, and spermatid differentiation. In another embodiment, inhibition of Bruce expression, activity or function is a means of inhibiting spermatid differentiation by increasing the sensitivity of differentiating spermatids to damage from caspase activity.

In one embodiment, the invention provides a method of inhibiting or abrogating spermatid differentiation, comprising the step of contacting a spermatogenic cell with an agent that inhibits or abrogates expression or activity of cyt-c-d, Sept4/ARTS, or Bruce in said cell, thereby inhibiting or abrogating spermatid differentiation. According to this aspect of the invention, in one embodiment, inhibition of cyt-c-d or Sept4/ARTS expression, activity or function inhibits caspase expression, activity or function. In another embodiment, inhibition of Bruce expression, activity, or function increases the sensitivity of differentiating spermatids to damage from caspase activity.

It is to be understood that the agents employed throughout, and methods of contacting cells with an indicated agent includes any embodiment herein described, and the spermatogenic cells will also be considered in the context of the embodiments listed herein.

Male Contraceptives:

Inhibition of spermatid differentiation provides, in another embodiment, a method of male contraception.

Male contraceptive agents for widespread use, without limitation upon their effectiveness and reversability has not yet been achieved. Though compounds inhibiting or arresting spermatogenesis have been explored for this purpose [see Bennett, J. P. (1974), Chemical Contraception, Columbia Press, New York, pp. 133-170; Davies, A. G. (1980), Effects of Hormones, Drugs and Chemicals on Testicular Function, Vol. 1, Eden Press, Westmont, pp. 123-164; and Jeffcoate, S. L. and Sandler, M. (Editors), Progress Towards a Male Contraceptive, John Wiley and Sons, Chichester, 1982], cytotoxic, neurotoxic or anti-metabolic effects, or untoward effects on libido, accessory sex glands and the male endocrine system (see Jeffcoate and Sandler, supra) render their application problematic. Moreover, the compounds that have been tested generally require fairly large daily doses and their long range effects remain unknown (see Shandilya, L., Clarkson, T. B, Adams, M. R., and Lewis, J. C. (1982). “Effects of gossypol on reproductive and endocrine functions of male cynomolgus monkeys (Macaca fascicularis)”, Biol. Reprod. 27:241-252).

In one embodiment of this invention, there is provided a method of male contraception, comprising contacting a spermatogenic cell in a subject with an agent that diminishes or abrogates caspase expression or activity in said spermatogenic cell.

Cell specific targeting of molecules is well described in the art, and may be accomplished, in one embodiment, via direct introduction to the tissue site, or, in another embodiment, via the generation of carriers specifically targeting a tissue site, following delivery parenterally. Agents mediating caspase inhibition may be delivered specifically to the testes, in another embodiment, for inhibition of spermatogenesis, without affecting other organs or systems.

Caspase activity plays a role in spermatogenesis in the mouse as well as the fly. Mice engineered to be deficient in Sept4/ARTS expression, a known caspase activator, are viable yet male-sterile, displaying cytoplasmic droplet sperm morphology (data not shown). Some caspase activity can be detected in testes of Sept4/ARTS −/− males, though overall caspase activity is diminished (data not shown). Moreover, the sperm of Sept4/ARTS −/+ heterozygous males exhibit phenotypic effects, which are related to the reduction of intracellular Sept4/ARTS concentration through the gene dosage effect phenomenon. The normal phenotype of the +/− mice in other body tissues indicates that spermatogenesis is more sensitive than other cellular processes to diminished caspase activation.

In one embodiment, suppressing spermatid differentiation is via supply of an agent suppressing caspase expression, activity or function. In one embodiment, small amounts of the agent suppressing caspase expression, activity or function are sufficient to suppress spermatid differentiation. In one embodiment, caspase inhibition is accomplished via an agent at a concentration that exclusively affects spermatid differentiation. In another embodiment, the agent is at a concentration that minimally affects processes other than spermatid differentiation.

In another embodiment, the invention provides a method of male contraception comprising contacting a spermatogenic cell in a subject with an agent that diminishes or abrogates expression or activity of cyt-c-d, Sept4/ARTS, or Bruce in said spermatogenic cell. According to this aspect of the invention, in one embodiment, diminishing or abrogating expression, activity or function of cyt-c- or Sept4/ARTS diminishes or abrogates caspase expression activity or function, thereby inhibiting spermatid differentiation. In another embodiment, diminishing or abrogating Bruce expression, activity, or function abrogates protection from the harmful effects of caspase activity, thereby inhibiting spermatid differentiation.

Cyt-c-d and Sept4/ARTS loss-of-function mutants were male sterile, presenting a sperm morphology consistent with experimental caspase inhibition and phenotypically indistinct from sperm morphology seen in the most common form of adult male infertility. No other phenotypic effects were observed (Example 8), highlighting specific roles for cyt-c-d and Sept4/ARTS in spermatid differentiation. Inhibition of cyt-c-d expression, activity or function, provides, in one embodiment, a highly specific means of male contraception. According to this aspect of the invention, cyt-c-d inhibition is accomplished with an agent as herein described.

In another embodiment, the invention provides a method for identifying an agent, which functions as a male contraceptive.

According to this aspect of the invention, the method of identifying an agent that decreases or abrogates the fertility rate of a male mammal comprises administering a candidate agent to a non-human male animal expressing a caspase, cyt-c-d, Sept4/ARTS or Bruce gene, wherein the agent alters caspase, cyt-c-d, Sept4/ARTS or Brucegene or gene product expression, activity or function, and the effect of the agent on the animal's fertility rate is determined. A decreased fertility rate indicates that the candidate agent functions as a male contraceptive.

Spermatogenic cells are sensitive to caspase concentration. Caspase inhibitors are currently in clinical trials for various degenerative conditions, including acute alcoholic hepatitis, stroke, sepsis. Diminished caspase expression, activity or function is readily discernable via detection of inhibition or abrogation of spermatid differentiation.

In another embodiment, there is provided a method of monitoring in vivo caspase inhibition in a male subject in need, comprising the steps of obtaining a semen sample from a male subject in need and determining the presence of spermatid differentiation in said semen sample, wherein diminished spermatid differentiation indicates in vivo caspase inhibition. Diminished spermatid differentiation, according to this aspect of the invention, functions as an indicator for bioavailability of a given caspase inhibitor in males.

Pest Control:

A requirement for caspase activation in spermatid differentiation is common to mammals and insects (Example 4). Caspase inhibition, either directly, or indirectly via disruption of cyt-c-d or Sept4/ARTS function in insects results in male sterility, with no affect on viability (Examples 7 & 8). In addition, protection of spermatogenic cells from the effects of caspase activity by dBruce protein plays a role in spermatogenesis, at least in insects (Example 10).

In another embodiment, inhibition or abrogation of expression, function or activity of cyt-c-d, caspase, Sept4/ARTS or Bruce can be exploited as a means of pest control.

According to this aspect of the invention, and in one embodiment, there is provided a method of arthropod pest control, comprising, contacting a spermatogenic cell of said pest with an agent that diminishes or abrogates caspase expression or activity.

In one embodiment, the arthropod is an insect. The term “insect” is used herein in its broad common usage to include spiders, mites, ticks and like pests which are not in the strict biological sense classified as insects. Thus, the usage herein conforms to the definitions provided by Congress in Public Law 104, the “Federal Insecticide, Fungicide, and Rodenticide Act” of 1947, Section 2, subsection h, wherein the term “insect” is used to refer not only to those small invertebrate animals belonging mostly to the class Insecta, comprising six-legged usually winged forms, as beetles, bugs, bees, flies, and so forth, but also to other allied classes of arthopods whose members are wingless and usually have more than six legs, as spiders, mites, ticks, centipedes, and wood lice.

In another embodiment, there is provided a method of rodent control, comprising, contacting a spermatogenic cell of said rodent with an agent that diminishes or abrogates caspase expression or activity. In one embodiment, the rodent is a mouse. In another embodiment, the rodent is a rat.

It is to be understood that all embodiments described herein, such as for contacting spermatogenic cells and agents for diminishing or abrogating caspase expression, activity or function, are to be applied for this aspect of the invention.

In another embodiment, according to this aspect of the invention, the agent can be used alone or in combination with an adjuvant in liquid, solid or gaseous form, as part of a composition. In another embodiment, the composition containing the agent may further comprise diluents, extenders, carriers and conditioning agents to provide a composition in the form of finely divided particulate solids, semi-solids, aerosols, solutions and dispersions or emulsions. The composition may comprise any suitable combination, as well.

The composition comprising the agent, according to this aspect of the invention, may also comprise attractants for the particular insect being controlled, representing another embodiment of the invention. For example, the agent can be applied to or admixed with attractants or baits such as sucrose, glucose, molasses, protein mixtures, powdered egg yolk, powdered milk, yellow corn grits, quincy granules, pumice granules, sex attractants, and the like.

The composition may further comprise an insecticide, as well. The term “insecticide” is defined herein to mean a compound, which kills insects. The term does not include compounds, which reduce fecundity of the housefly. Numerous such compounds are known in the art, particularly carbamate and organophosphate insecticides.

Numerous devices for the delivery of such agents to arthropods are well known in the art (see for example, U.S. Pat. No. 4,666,767 or U.S. Pat. No. 4,639,393), and their implementation with agents and compositions of the invention, as described herein, represent additional embodiments of the invention.

The invention may also be used to identify additional chemosterilant agents.

In another embodiment, this invention provides a method of identifying an agent that decreases or abrogates the fertility rate of a male arthropod comprising administering a candidate agent to a male arthropod expressing a caspase, cyt-c-d, Sept4/ARTS or Bruce gene, wherein the agent alters caspase, cyt-c-d, Sept4/ARTS or Bruce gene or gene product expression or activity. Decreased male arthropod fertility indicates that the candidate agent functions as a chemosterilant.

The following are meant to provide materials, methods, and examples for illustrative purposes as a means of practicing/executing the present invention, and are not intended to be limiting.

### EXAMPLES

### Materials and Methods

Fly Strains and Rearing Conditions

The Canton S and the yw strains served as wild-type controls. The fuzzy onions mutant alleles (fzo1 and fzo2) were obtained from M. T. Fuller (Stanford University School of Medicine, California, USA) and were crossed to each other to allow recovery of trans-heterozygous progeny. The jaguar (jar1), purity of essence (poe1), hephaestus (heph2), blanks (bln1), Df(2L)H20, 1(2)k13905 and D2-3 ‘jumpstart’ lines were obtained from the Bloomington Stock Center. The EP(2)2305 and EP(2)2049 lines were obtained from Exelixis and Szeged Drosophila Stock Centre, respectively. The strain 1(2)k11502 was used as a hac-1 enhancer trap as previously described (Zhou et al., 1999). The dBruce alleles were previously isolated in our lab in a genetic screen for cell death modifiers (Agapite and Steller, unpublished results). dBruce81-e corresponds to an in frame deletion of amino acids 234-411, which leads to the complete removal of the BIR domain. To generate a GAL4 driver line for expression in late spermatogenesis stages, the GAL4 coding sequence flanked by hsp70 UTR sequences was excised from the vector pGaTB (Brand and Perrimon, 1993) and subcloned into the CasperR-HSP83 vector (Hicks et al., 1999) downstream of the hsp83 promoter. Transgenic lines for HSP83-Gal4 were obtained and crossed to the UAS-p35 strain (Hay et al., 1994) using standard fly genetics.

Antibodies

The following primary antibodies were used to stain squashed testes preparations: anti-lamin Dm0 (mAbADL84 from P. Fisher, 1:100), rabbit CM1 antiserum (IDUN Pharmaceuticals Inc., 1:1000), rabbit anti-FL-DRICE and rabbit anti-active DRICE (from B. Hay, 1:500 and 1:1000, respectively) (Dorstyn et al., 2002), anti-cytochrome c (mAb2G8, from R. J. Jemmerson, 22 mg/ml) (Varkey et al., 1999), Guinea pig anti-FL-DRONC (from H. D. Ryoo, unpublished, 1:2000), and rabbit anti-β-galactosidase (from Cappell, 1:1000). All secondary antibodies were purchased from Jackson ImmunoResearch Laboratories and were diluted 1:500.

Antibody Staining

At least 20 testes were examined for each experiment described. Testes of young adults were dissected in testis buffer (TB) which consisted of: 10 mM Tris-HCl, pH 6.8, 183 mM KCl, 47 mM NaCl, 1 mM EDTA, and 1 mM PMSF. Dissected testes were transferred to a 2.5 ml droplet of TB placed on a siliconized coverslip (GOLD SEAL), opened using thin forceps, and sandwiched with a poly-L-lysine-coated slide. The sandwich was frozen in liquid nitrogen, the coverslip was removed with a razor blade, and the slide was placed in ice-cold absolute ethanol. The slides were drained and a hydrophobic ring surrounding the opaque tissue was drawn using a PAP PEN (Zymed Laboratories Inc.). The tissue was fixed in 4% formaldehyde in PBS for 20 minutes, rinsed twice with PBS for 5 minutes, incubated in PBT (PBS+0.1% Triton X-100) for 30 minutes, and rinsed twice again. The fixed testes were then blocked with PBS/BSA (1% BSA in PBS) for 45 minutes, incubated with primary antibody (diluted in PBS/BSA) within the hydrophobic ring overnight at 40° C. inside a humid chamber, and rinsed twice for 5 minutes in PBS. Testes were incubated with the secondary antibody (diluted in PBS) for 1 hour at room temperature, rinsed twice in PBS for 5 minutes, incubated with 1 ng/ml TRITC-phalloidin (Sigma) in PBTw (PBS+0.1% Tween 20) for 1.5 hour in 37° C. in a humid chamber, rinsed once for 15 minutes at room temperature, and mounted in Vectashield mounting medium with DAPI (Vector Laboratories). For AO staining, testes were dissected in TB, incubated for 5 minutes in fresh 1.6×10-6 M AO (Sigma) in TB, rinsed briefly in TB and mounted in TB. All pictures were taken by using confocal microscopy (Zeiss Axioplan 2).

The immunocytochemistry procedure was essentially as described in (Gonczy et al., 1992). Twenty testes of each line from 0-1-day-old adults were dissected in TB, fixed for 20 minutes in 4% formaldehyde in PBX (PBS plus 0.1% Triton X-100) and washed three times for 10 minutes in PBX The samples were blocked for 90 minutes in Blotto (5% powdered milk in PBX) and incubated overnight at 40° C. with 1:1000 rabbit CM1 antiserum in Blotto. The rest of the experiment was carried out at room temperature. The samples were washed three times for 10 minutes in PBX, incubated for 60 minutes with 1:50 biotinylated universal antibody (Vector Laboratories) in Blotto, and washed three times for 10 minutes in PBX. Signal detection was carried out with a Vectastain Universal Elite ABC kit (Vector Laboratories): during the last washes the A and B reagents were incubated together (ABC reagent, 1:50 avidin DH+1:50 biotinilated horseradish peroxidase H in PBX) for 30 minutes. The samples were incubated with the ABC reagent for 30 minutes and washed 5 minutes with PBX. Diaminobenzidine tetrahydrochloride (DAB) solution was made as follows: one brown and one white tablet (SIGMA Fast DAB Tablet Sets) were dissolved in 1 ml PBX for 20 minutes. The samples were incubated in DAB solution until desired stain intensity developed, washed with 120 mM Tris-Cl pH 7.6, and mounted in a drop of 70% glycerol in PBS. Samples were examined by Nomarski optics on a Zeiss Axiophot.

RNA Isolation and Northern Blotting

Total RNA was extracted from one hundred adult male flies by using the TRIZOL Reagent according to the manufacture's instructions (Invitrogen). The average yield was 0.8 mg RNA per 100 mg starting material. Poly(A) RNA was isolated (PolyATract III, Promega), and 2.5 mg poly(A) RNA were loaded per lane. Blotting was carried out according to standard protocols, and the nylon membrane (Amersham) was hybridized to the probe in ULTRAhyb buffer (Ambion). The probe was prepared by using a fragment of about 200 bp from the 3′ UJTR region of cyt-c-d (BDGP's EST clone LP05614 cleaved with BseRI and XhoI), and 32P-labaled using the 5′-end labeling kit (Amersham). The membrane was washed briefly and re-hybridized to a RP49 probe as a loading control.

Testes Cultures

Testes were dissected in a drop of culture media [10% Fetal calf serum, Modified Shields and Sang M3 medium (-bicarbonate; Sigma) with 1× penicillin/streptomycin (GIBCO BRL)]. Each isolated testis was then placed in separate well within a 96 well plate (Corning) with 100 ml preheated (25° C.) of culture media, along with 30 mm of the caspase inhibitor Z-VAD(OMe)-FMK (Enzyme Systems Products). This compound was diluted in DMSO from a concentrated stock. Control incubations using DMSO alone (typically 0.1%-0.2%) were performed in parallel. The plates were incubated in a 25° C. incubator for 24-26 hours. Following incubation, testes were fixed and stained as indicate above.

Fertility Test

20 young adult males of each genotype were placed individually in separate vials with food at 25° C. Each vial was supplemented with 3 wild-type virgin females. The flies were allowed to mate for a week and the number of offspring was determined.

P-Element Excisions and Isolation of Fertile Revertants

To generate revertants from either the bln1 or the EP2305 strains, the p[Z] and EP transposons were excised using the transposase-producing D2-3 ‘jumpstart’ strain (Robertson et al, 1988). For the bln1 strain we used the following excision scheme: bln1/bln1 females were crossed to Sco/CyO; D2-3, ki males, and progeny of the bln1/CyO; D2-3, ki genotype were crossed to the original bln1/CyO strain. Groups of 5 males, which contain a potential excised allele over the bln1 allele, were allowed to copulate with wild-type females for 4 days for a fertility test. Then, testes of fertile groups were dissected and analyzed for the presence of active-drICE and proper individualization structures. To isolate revertants of the EP2305 strain, orange eye flies of the genotype w[−]; P{w[+mC]=EP}EP[2305]/CyO; D2-3, ki were crossed as singles to flies of the w[−]; Sco/CyO strain and progeny with white eyes, w[−]; */CyO (*=excised transposon), were crossed as singles to the original EP2305/CyO strain. From each cross, w[−]; */EP2305 males were subjected for sterility test, and their white-eye siblings were kept as a stock for further analysis as described above. The same protocol was used to isolate revertants of the EP2049 strain.

### Example 1

### Apoptosis Occurs During Spermatid Individualization

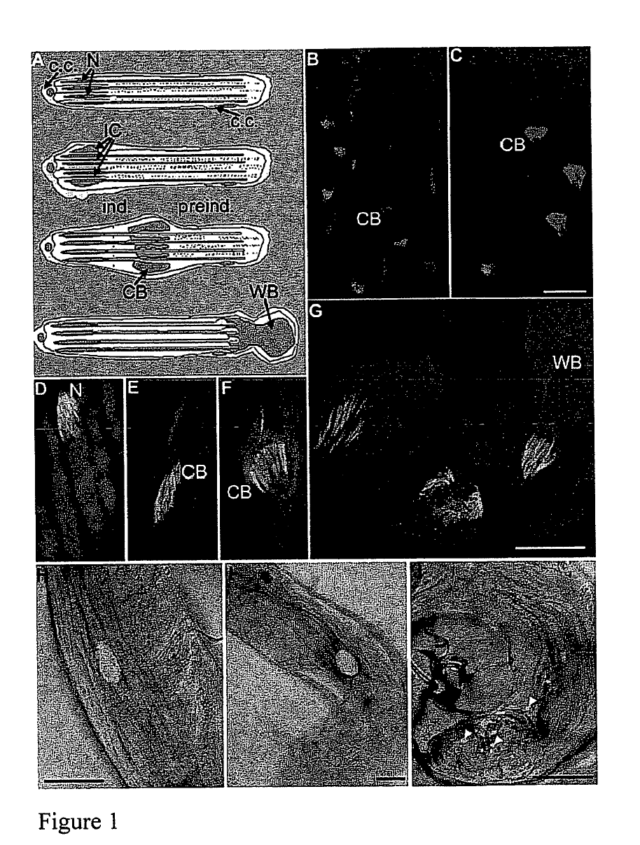

Spermatogenesis in Drosophila melanogaster takes place within individual units known as cysts. Each cyst contains 64 spermatids that remain initially connected after meiosis via cytoplasmic bridges. All spermatids within a cyst differentiate synchronously and undergo dramatic cellular and morphological changes. During terminal differentiation, the round-shaped spermatids were transformed into thin, approximately 2 mm long, spermatozoa with highly elongated, “needle-shaped” nuclei. During the final stage of spermatogenesis, termed individualization (schematically depicted in FIG. 1A, based largely on the data of Tokuyashu et al., 1972), the cytoplasmic bridges were disconnected and most of the cytoplasm was expelled, leading to individual sperm.

The schematic diagram, (FIG. 1A) depicts a single cyst with four representative spermatids at increasingly advanced developmental stages (from top to bottom). The first cyst contains elongated spermatids prior to individualization. In the second cyst, assembly of the individualization complex (IC) has begun around the nuclei. The IC progresses caudally from the nuclear region to the end of the tail (in this figure from left to right). The third cyst has a cystic bulge (CB), which contains the progressed IC and the cytoplasm of the post individualized part of the spermatids. At this point, many vesicles and organelles are present in the CB. Finally, most of the spermatids' syncytial cytoplasm is removed into the waste bag (WB). The WB is pinched off from the base of the cyst and eventually degrades. Each sperm is now surrounded by its own membrane and is largely devoid of cytoplasm and organelles (with the exception of a single giant mitochondrion).

The individualization process involved the assembly of a cytoskeletal-membrane complex, referred to as the “individualization complex” (IC), which contained actin as its major cytoskeletal component. The IC was detected by staining with Phalloidin that binds actin (FIGS. 1B and 1C) (Fabrizio et al., 1998). In addition, we observed that lamin Dm0 translocated as a component of the IC and was thus a useful marker of the IC (FIGS. 1D-G).

In squashed testes preparations, bundles of wild-type elongated spermatids (blue) are depicted either before IC assembly (B), associated with an IC (B and D, IC in pink, (merged Red (TRITC-phalloidin, staining actin) and Blue Channels), or after the IC translocation (E, IC in green, FITC-tagged anti-Ig-anti Dm0, labeling laminin). The IC was assembled at the nuclear end of the cyst and subsequently translocated caudally along the entire length of the spermatid bundle, expelling most of the cytoplasm in the process. The discarded cytoplasm accumulated in a membrane-enclosed structure, termed the waste bag (WB, FIGS. 1A, 1G and 1I) (Tokuyasu et al., 1972; Fabrizio et al., 1998). The WBs eventually underwent fragmentation and subsequent degradation (FIG. 1J).

The CB size increased with spermatid individualization progression; small CBs are evident right after the detachment of the IC from the vicinity of the nuclei (E, IC in green). As the IC progresses, the CB increases volume (C (IC in red), and F (merged photo with lamin in green and actin in red IC). Finally, the CB reaches the end of the spermatids becoming a large WB (G, IC in green).

To investigate the possible occurrence of apoptosis during Drosophila sperm differentiation, we stained live wild-type testes with the vital dye acridine orange (AO), which specifically detects apoptotic cells. AO staining was observed in two major locations: in oval structures of variable size running along the testis, and in randomly scattered fragments at the base of the testis, adjacent to the seminal vesicle (FIGS. 1H-1J). A closer examination revealed that the oval structures represented intact cystic bulges (CBs) and WBs (FIGS. 1H and 1I), whereas the staining of scattered, fragmented structures corresponded to the degrading WBs. The staining of the CB and WB with AO in spermatids undergoing individualization suggested that the apoptotic program was activated in the late stages of sperm differentiation, and that CB and WB resemble apoptotic corpses without nuclei.

### Example 2

### Activation of an Effector Caspase at the Onset of Spermatid Individualization

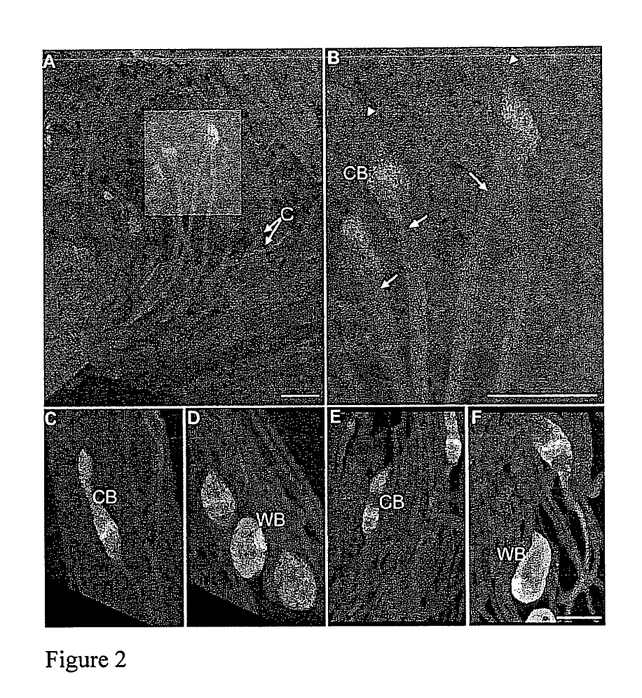

A key feature of apoptosis is the activation of caspases. Once activated, caspases mediate apoptosis by cleaving specific intracellular proteins, including proteins of the cytoskeleton and cytosol. To investigate whether Drosophila caspase activation occurred during the individualization process, testis preparations were stained with the CM1 antibody. CM1 is a polyclonal rabbit antiserum raised against the active form of mammalian caspase 3. This antibody cross-reacts with the caspase-3-like Drosophila effector caspase drICE and stains apoptotic cells in situ. In addition, we also used an antibody that was raised specifically against the active form of drICE. Using either antibody, active drICE was detected immediately after a mature IC was formed. At this stage, a 2 mm segment from the bundled nuclei to the distal end of the spermatids exhibited strong immunoreactivity with either antibody (FIGS. 2A and 2B). During the caudal translocation of the IC, active drICE became completely depleted from the newly individualized portion of the spermatids. The staining remained abundant, however, in the pre-individualized portion of the spermatids with the highest levels within the CB (FIGS. 2B, 2C and 2E). At the end of this process, the newly formed WBs contained high levels of active drICE (FIGS. 2D and 2F). drICE levels declined as spermatids coiled and eventually the WBs were degraded at the base of the testis (FIG. 1J). These observations indicated that caspase activation and apoptosis-like events were intimately associated with terminal spermatid differentiation in Drosophila.

### Example 3

### Inhibition of Caspase Activity Impedes Spermatid Individualization

In order to determine whether caspase activation was necessary for spermatid differentiation, caspase activity was inhibited both in cultured testes and in vivo. Under in vitro culture conditions, each cyst accomplishes individualization within 12 hours. Caspase inhibition of IC caudal movement was determined in vitro, using isolated pairs of testes from young adult flies (n=10). One testis of a pair from the same individual was cultured in the presence of the caspase inhibitor Z-VAD, while the other testis served as a control. Z-VAD blocks the activity of drICE in vitro, and drICE-induced cell death of Drosophila S2 cells. Testes cultured with the caspase inhibitor demonstrated advanced CBs that appeared flat (FIG. 3B) as compared to the full, oval shape of CBs of controls (FIG. 3A), indicating that caspase activity was involved in the efficient collection of cytoplasm into the CBs. (Nuclei were visualized with DAPI (blue), IC's with phalloidin (red), and active drICE with the CM1 antibody (green)).

IC translocation was also defective in the absence of caspase activity. In controls, only a few early ICs were associated with nuclear complexes since ICs translocate caudally during spermatid differentiation (FIG. 3C). In contrast, ICs cultured with caspase inhibitor predominantly localized around nuclei (FIG. 3D), indicative of a defect in IC-translocation. Therefore, caspase activity appears to be involved in proper IC movement and removal of bulk cytoplasm from differentiating spermatids.

In order to determine the effects on spermatogenesis in vivo, the baculoviral broad-spectrum caspase inhibitor p35 was expressed in male gonads via the GAL4ΔUAS expression system (Brand and Perrimon, 1993). Transgenic flies were generated that carry the GAL4 gene fused downstream of the Drosophila Hsp83 promoter, which directs strong expression in germ cells throughout spermatogenesis. The Hsp83-gal4 driver line was crossed to the UAS-p35 line and male progeny were subjected to a fertility test. While the parental lines that bear two copies of either of the constructs alone were fertile, male progeny with both the driver construct and the UAS-p35 construct were sterile. Cytological analyses of these males revealed that IC translocation was severely impaired (FIG. 3E). Although the initial assembly of the IC appeared normal (data not shown), the advanced IC cones were scattered throughout the length of the cyst, and the cytoplasm failed to collect to form a normal CB. Instead of becoming concentrated into a single, large CB, the cytoplasm was distributed over many small bulges throughout the cyst (FIG. 3E). (Note that previous reports of the inhibition of drICE by p35 failing to block CM1-staining were confirmed. In contrast, cysts in both parental lines (Hsp83-gal4 driver and UAS-p35) appeared completely normal (data not shown). These observations demonstrated that caspase activity is involved in proper spermatid individualization and male fertility. In particular, it appears that caspases are involved in facilitating the movement of the IC and in collecting the spermatid cytoplasm in the cystic bulge.

### Example 4

### Caspase Activity is Involved in Both Drosophila and Mammalian Spermatid Differentiation

Intracellular bridges between spermatids and the bulk of the spermatid cytoplasm must be eliminated during Drosophila and mammalian spermatogenesis. In mammals, the cytoplasm collects in the residual body (RB), which is functionally homologous to the WB in Drosophila (FIG. 3F). Mammalian RBs have been reported to display apoptotic features and active caspase-3 was found to be present in residual bodies in the testes of mice (Kissel, Arama and Steller, unpublished results), similar to its localization within WBs in Drosophila.

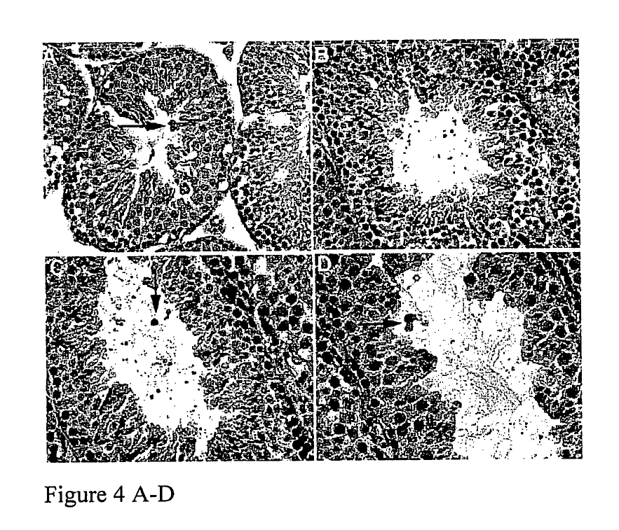

Caspase inhibition produced abnormal spermatozoa with accumulated residual cytoplasm (FIG. 3E), morphologically comparable to one of the most commonly seen abnormalities of human spermatozoa, known as cytoplasmic droplet sperm (FIG. 3G), which is characterized by immature sperm retaining cytoplasm with a residual volume of more than one third of the sperm head area. Caspase-3 is activated during mouse spermatid terminal differentiation (FIG. 4, panels A-D, with staining localization indicating that caspase 3 is activated at a very late stage during spermatogenesis, in the lumen of the seminiferous tubule. Specifically, strong staining was detected in the residual bodies (RB) that were pinched off from elongated spermatids into the lumen of the tubules (black arrows). Therefore, murine residual bodies share functional and biochemical similarities with the waste bag (WB) of Drosophila.

The sperm of homozygous Sept4/ARTS knockout mice exhibited a cytoplasmic droplet morphology (F), which was not seen in sperm from wild-type mice (E). These mice had no obvious abnormalities other then sperm morphology, indicating Sept4/ARTS involvement in spermatid differentiation.

### Example 5

### Caspase Activation at the Onset of Spermatid Individualization is Independent of Terminal Differentiation

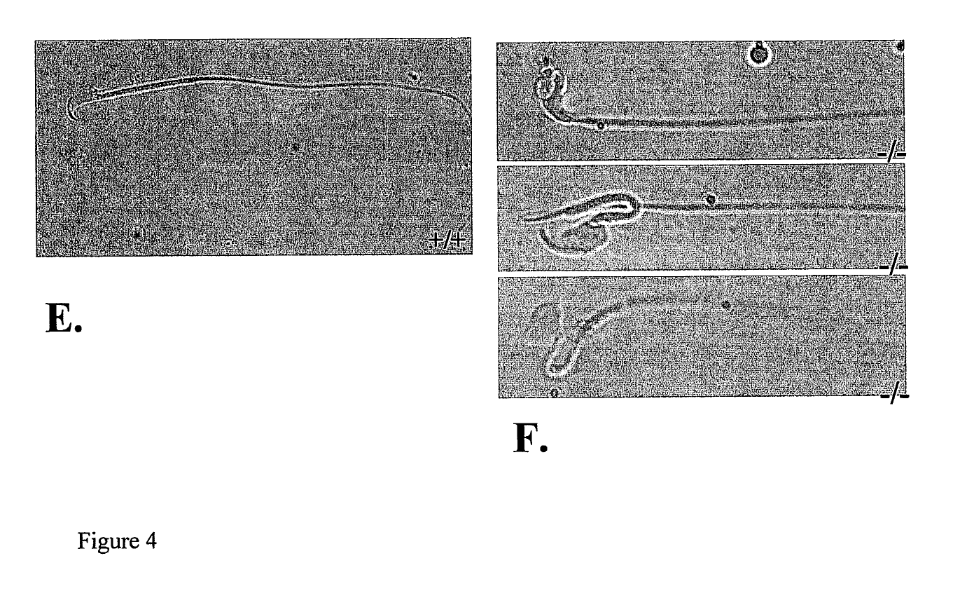

To distinguish whether caspase activation at the onset of spermatid cytoplasm proteolysis is the cause or the consequence of sperm cell differentiation, we examined mutants that disrupt the individualization process. In purity of essence (poe1) spermatids the ICs are significantly reduced (arrowheads in FIG. 5A), and often are not detected at all (arrows in FIG. 5A). However, active-caspases are clearly detected in elongated spermatids that lack the IC, suggesting that caspase activation at the onset of individualization is independent of IC assembly. In jaguar (jar1) male flies, IC assembly and movement are impaired due to a mutation in a class VI myosin. Interestingly, in jar1 flies active drICE could be only detected in mature cysts, which contain needle-shaped and condensed nuclei (FIG. 5B). Therefore, the completion of spermatid morphogenesis rather than the assembly of a functional IC triggers drICE activation.

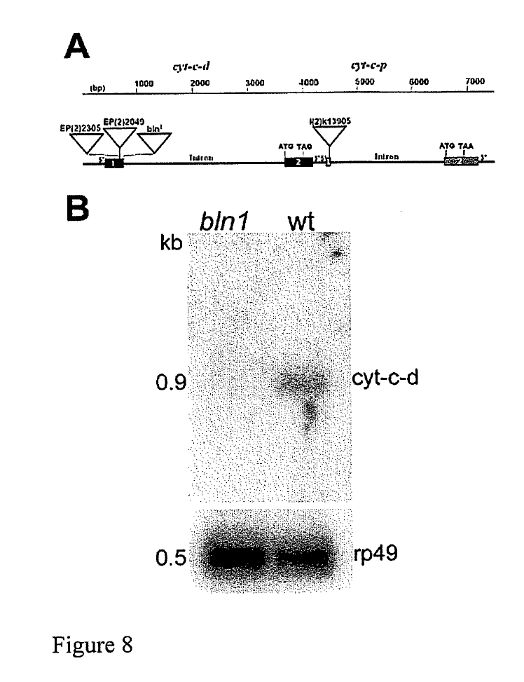

To determine whether caspase activation is dependent upon the completion of nuclear morphogenesis, we examined testes of fuzzy onions (fzo) mutant flies. fzo flies are defective in the process of mitochondrial fusion during spermatogenesis, and hence they arrest at mid-spermatid-elongation stage. We observed that active drICE accumulated in several immature elongating spermatid cysts containing undifferentiated nuclei (FIG. 5C). In addition, 11 other mutants that cause severe defects in individualization were analyzed (see below), and in all of them we detected cysts that displayed intensive CM1 staining (see, for example, FIG. 9E). Therefore, we conclude that drICE activation is independent of nuclear maturation.

### Example 6

### Components of the Apoptosome are Expressed During Spermatogenesis

Another aspect of sperm terminal differentiation is the elongation of the mitochondria along the entire length of the spermatid. Mitochondria play an important role in the activation of the apoptosome, a multiprotein complex that includes caspases, Apaf-1 and cytochrome c in mammals, with orthologues identified in Drosophila, and therefore, their expression during Drosophila spermatogenesis was determined.

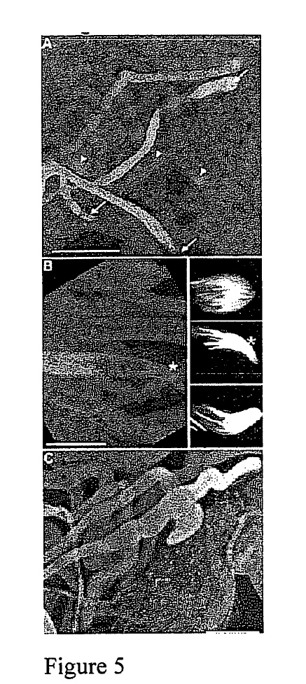

Drosophila Apaf-1 homologue hac-1/dark/dapaf expression during spermatogenesis, was determined via b-galactosidase distribution in testes of the enhancer-trap line 1(2)k11502, which mimics the mRNA hac-1 expression. As shown in FIG. 6C, hac-1 accumulated in late primary spermatocytes.

A Drosopholia caspase 9 orthologue, Dronc was expressed along the length of elongated spermatids (arrowheads in FIGS. 6A and 6B). During IC formation Dronc expression was punctate, yet disorganized As the IC progressed caudally, Dronc expression increased within the CB (arrowhead). Following IC translocation, Dronc was expressed in the post-individualized portion of a cyst, though demonstrably less. Eventually, Dronc staining disappeared from mature spermatozoa (arrow in FIG. 6A).

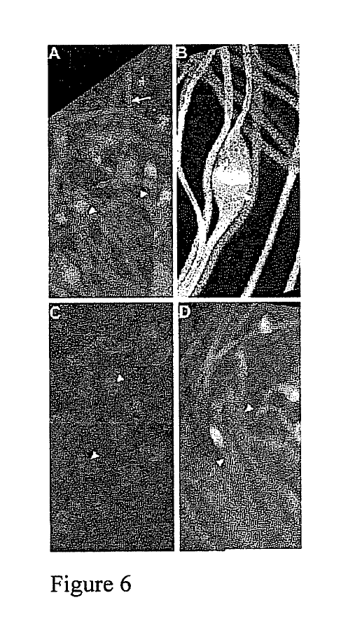

Drosophila pro-drICE accumulated in primary spermatocytes and was uniformly distributed in pre-individualized elongated spermatids (FIG. 6D), as detected by a polyclonal antibody specific for Dronc (FIG. 7A), which stained ectopically expressed dominant negative (DN) Dronc in Drosophila wing imaginal discs (FIG. 7B). A significant proportion of cellular Dronc and drICE localized near mitochondria. Moreover, Dronc is recruited to an apoptosome-like complex in cell extracts supplemented with cytochrome c, in vitro. Thus Drosophila apoptosomes were formed at mitochondrial surfaces.

### Example 7

### A Specific Mitochondrial Cytochrome C Affects Male Sterility

In mitochondrial induction of apoptosis, several pro-apoptotic proteins are released into the cytosol in response to apoptotic stimuli, such as cytochrome c. In order to determine whether there is a requirement for mitochondrial cytochrome c in sperm cell differentiation in Drosophila, cytochrome loss-of-function mutants were utilized. Drosophila contains two closely linked but distinct cytochrome c genes, termed cyt-c-d and cyt-c-p (also known as DC3 and DC4, respectively). The cyt-c-p gene encodes the major form of cytochrome c and is expressed at much higher levels than cyt-c-d.

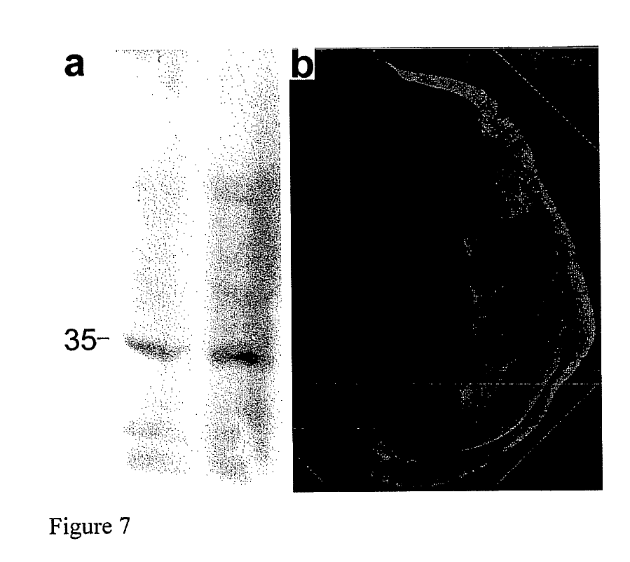

Searching FlyBase identified 3 independent P-element insertional mutations located in cyt-c-d (bln1, EP²³⁰⁵ and EP²⁰⁴⁹), and one insertion in cyt-c-p. The genomic organization of the two cytochrome c genes and the position of P-element insertions are shown in FIG. 8A. The predicted ORF for cyt-c-d encodes a protein of 105 amino acids. The three cyt-c-d insertions were identified in the 5′ UTR of exon-1. The p[Z] element of the bln1 allele is inserted 2 base pairs (bp) upstream of the last nucleotide of exon-1, in chromosomal region 36AB, while the EP²³⁰⁵ and EP²⁴⁰⁹ insertions are 83 bp and 77 bp, respectively, upstream of the last nucleotide of exon-1. The bln1 insertion caused male but not female sterility. Sterility tests and complementation analyses revealed that both EP alleles are also male and not female sterile, and that they fail to complement the bln1 allele. In addition, we generated bln1/Df(2L)H20 trans-heterozygotes and found that they are also viable but male-sterile.

In order to verity that the mutant phenotypes were caused by transposon insertions, Northern blots probed with an oligonucleotide specific for the unique 3′ UTR of cyt-c-d identified one band of the predicted size in wild-type, but no transcript in bln1 mutants (FIG. 8B). Therefore, bln1 appears to be a complete loss-of-function allele for cyt-c-d.

P-element insertion in cyt-c-p, however, was homozygous lethal, and lethal in trans to a deletion that includes both cytochrome c loci, DF(2L)H20, and thus is not specifically related to male sterility, as is the case for cyt-c-d.

### Example 8

### Cyt-c-d Affects Fertility via Caspase Activation

In mitochondrial induction of apoptosis, cytochrome c is released into the cytosol, whereupon it binds to and activates Apaf-1, which in turn leads to the activation of caspase-9. No comparable role for mitochondrial factors in caspase activation has yet been established in invertebrates, however, and several reports have actually argued against a role for cytochrome c in caspase activation in Drosophila, since no release from mitochondria was seen in previous studies, and RNAi experiments in SL-2 cells produced negative results as well.

In order to determine whether there is a requirement for cytochrome c in caspase activation in Drosophila, the cytochrome c loss-of-function mutants were utilized. Testes from all cyt-c-d homozygous mutant alleles and all trans-hetyerozogyous combinations were probed for expression of active drICE via CM1 antibody staining.

No CM1 immunoreactivity was evident in bln1 homozogotes or bln1/DF(2L)H20 flies (FIGS. 9B, 9D and 9F), this despite that bln1 testes contained elongated spermatids with an intact IC and with the characteristic needle-shaped nuclei typical of advanced stages of spermatid differentiation (arrowheads in FIG. 9D). Nevertheless, while in wild-type the IC translocates caudally (arrows in FIGS. 9A and 9C), in bln1 mutants, the IC does not separate from the nuclei (FIG. 9D). EP²³⁰⁵ and EP²⁰⁴⁹ homozygotes were weakly hypomorphic as they demonstrated occasional CM1 staining and more progressed ICs, but all allelic combinations were male-sterile, although we observed some occasional escapers in EP^{2049 -/-} and EP²³⁰⁵/EP²⁰⁴⁹ flies (data not shown).

Hephaestus (heph²) mutants showed strong immunoreactivity with CM1 despite other severe defects in the individualization process (E), as did ten other mutants with defects in spermatid individualization (data not shown). Hephaestus is the fly homolog of the human gene PTB/hnRNP I protein, which has been shown to regulate alternate splicing of an important exon of caspase-2 and therefore may play a role in the positive regulation of caspase activity. Staining with mAB 2G8 (FIG. 9G), which detects cytochrome c only in apoptotic cells, stains the mitochondria of round spermatids (arrowheads) and then acquires a punctate expression pattern in elongated spermatids typical of mitochondrial protein expression at this stage (FIG. 9H) (Note that the giant mitochondrion of round spermatids can be easily visualized by DAPI due to staining of the mitochondrial DNA. Scale bars 50 μm).

All P-element revertants generated were fertile and demonstrated normal CM1 staining and individualization, confirming that the phenotypes reported were caused by transposon insertions in the cyt-c-d locus.

Thus a requirement for cyt-c-d for caspase activation at the onset of spermatid individualization was demonstrated. Loss-of-function mutants for cyt-c-d are homozygous viable but male-sterile. Significantly, these mutants were defective in drICE activation and failed to exclude bulk cytoplasm, producing phenotypes virtually identical to the ones resulting from the application/expression of caspase inhibitors.

### Example 9

### Differential Functions for Drosophila Cyt-c-d and Cyt-c-p

The cyt-c-p gene is located 241 bp downstream of cyt-c-d, contains two exons and one intron, and is predicted to encode a protein of 108 amino acids. The 1(2)k13905 allele is a P-lacW insertion in the first nucleotide of cyt-c-p intron and is a recessive embryonic lethal. Complementation analysis revealed that the bln1 cyt-c-d allele complemented the lethality of the 1(2)k13905 allele, and that the latter complemented the sterility of bln1. 1(2)k13905/DF(2L)H20 trans-heterozygotes are also recessive embryonic lethal, demonstrating that this phenotype is due to the loss of cyt-c-p function.

Testes probed for cytochrome c expression with the mAB 2G8 demonstrated increased immunoreactivity in early elongating and elongated spermatids of both wild type and bln1 mutants (FIGS. 9G and 9H and data not shown). The increased staining may be a function of expression of the major form of cytochrome c, encoded by cyt-c-p.

Both sterility and cellular phenotype map to the cyt-c-d gene, since all alleles failed both to complement each other and a deletion of this region. Furthermore, the different mutants can be arranged into an allelic series with respect to CM1 staining, ranging from no caspase activation (bln1/bln1 and bln1/Df) to weak caspase activation in EP^{2049 -/-} and EP²³⁰⁵/EP²⁰⁴⁹ mutants, indicating a role of the cyt-c-d gene for caspase activation during spermatogenesis in Drosophila. Cyt-c-p encodes the major fly cytochrome, important for the electron transport chain during aerobic respiration, and cyt-c-d has reportedly no function in respiration. Consistent with an essential role of cyt-c-p in respiration, a P-element insertion into this locus (FIG. 8A) resulted in recessive lethality (data not shown). Likewise, targeted gene inactivation of the murine cytochrome c gene causes very early embryonic lethality, and this has precluded functional studies on the role of cytochrome c for caspase activation during normal development in mammals.

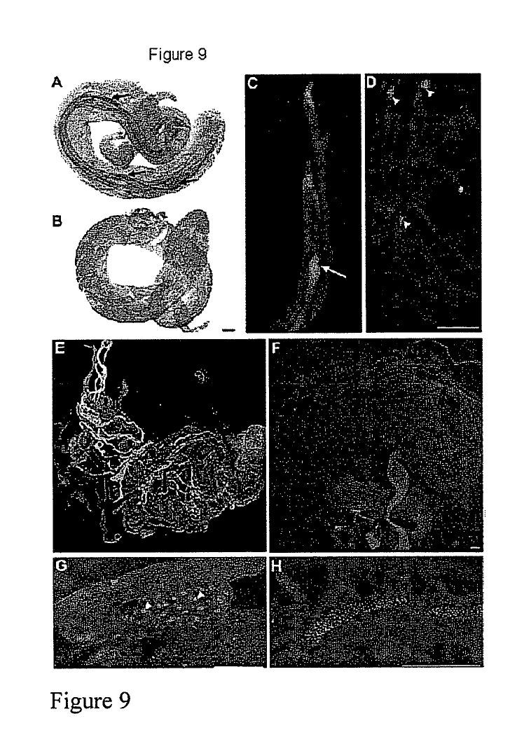

Insertion of the P-element also precluded cyt-c-d expression (FIG. 8B, 10A). Cyt-c-d effects on male fertility were further demonstrated with the recovery of revertants for cyt-c-d P-element insertions, wherein drICE-activation and individualization was restored (FIG. 10B), which was indistinguishable from the wild type.

Based on the results presented herein, the two cytochrome c genes in Drosophila fulfill distinct functions in respiration (cyt-c-p) and caspase-activation/apoptosis (cyt-c-d), respectively. Previous arguments against a role of cytochrome c for caspase activation in Drosophila were largely based on the failure to detect release of cytochrome from mitochondria. However, because cyt-c-d is expressed at much lower levels than cyt-c-p it would be virtually impossible to detect the release of the relevant protein in the absence of highly specific antibodies. Furthermore, since cyt-c-d null flies are viable and, apart from male-sterility, have no obvious anatomical defects, it is unlikely that this gene is broadly required for the activation of apoptosis. A complete block of apoptosis in Drosophila interferes with normal embryogenesis, and mutants with significantly reduced apoptosis can be viable but are phenotypically abnormal. Therefore, the main function of the cyt-c-d gene appears is in caspase activation during spermatid differentiation.

### Example 10

### Protection of the Sperm Nucleus

Effector caspases, such as drICE, Dcp-1 and caspase-3, which have been shown to participate in spermatid maturation, also cleave a variety of nuclear targets, including lamins, I-CAD, and PARP, which is potentially damaging to the sperm nucleus. For spermatid maturation, the sperm nucleus must be protected against their lethal activity. One candidate for such a protective function is dBruce, the Drosophila orthologue of mammalian Bruce/Apollon. Bruce/Apollon proteins are E2 ubiquitin conjugating enzymes that are thought to inhibit apoptosis. dBruce contains a BIR domain, a motif found in IAPs, which may mediate its binding to caspases or Reaper/Hid/Grim-like (RHG) proteins, though the data does not support RHG-proteins as direct targets for dBruce. Screens for genetic modifiers of Reaper-induced apoptosis identified 13 alleles of the Drosophila IAP-related gene, dBruce (Agapite and Steller, unpublished results), among which alleles were homozygous viable but male-sterile, behaving as loss-of-function alleles. Most of the dBruce mutations affected either the BIR or ubiquitin-conjugating (UBC) domains (data not shown).

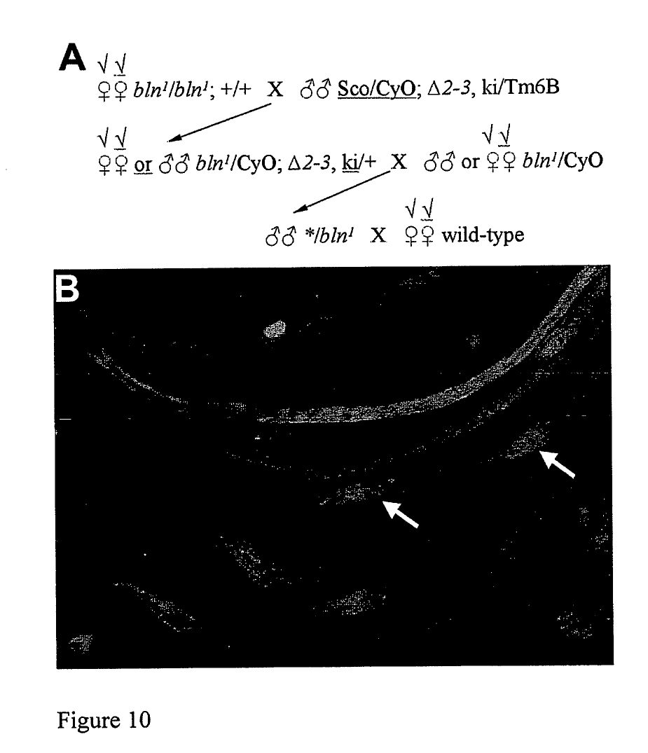

In dBruce −/− mutants, nuclear hypercondensation and degeneration occurred, indicative of excessive caspase activity (FIG. 11). Whereas nuclei in wild type acquired a highly elongated, needle-shaped morphology, nuclei in dBruce −/− mutants appeared much more highly condensed, rounded and scattered throughout the cyst. In addition, many nuclei stained very faintly, and eventually they degenerated and no functional sperm was formed. These observations are consistent with a role of dBruce in protecting spermatids against excessive caspase activity and death, via their direct binding to and degrading caspases. Limiting dBruce inhibition of caspase activity may be spatially restricted during spermatogenesis, for example, by localizing dBruce to protected compartments, or by local caspase activation within an affected compartment. The latter is supported by the strong CM1 staining observed distal to nuclei, exclusively in the cytoplasmic compartment. Local release of cyt-c-d from mitochondria, known to undergo dramatic morphological changes only in the post-individualized portion of the cyst, provide further support for the latter, as well.

### Example 11

### sept4 Null Mice are Male Sterile

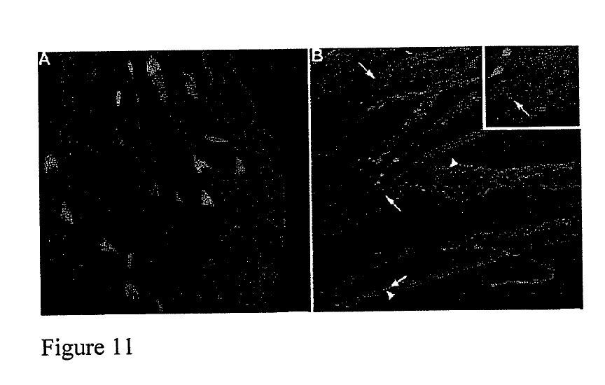

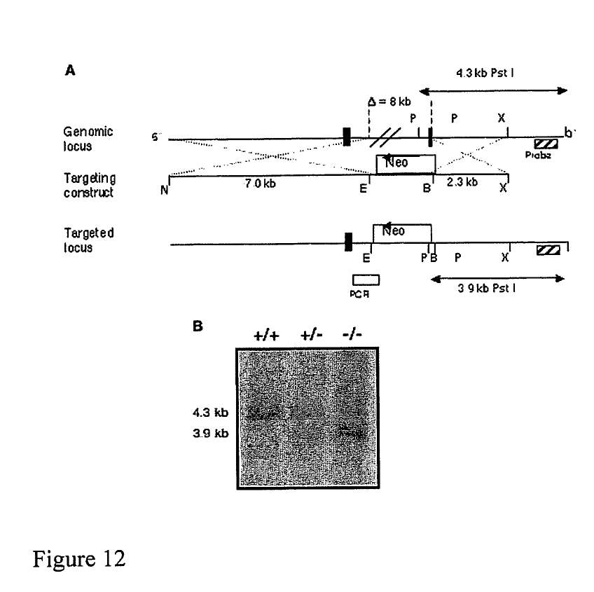

In order to further delineate the role of sept4 in sperm differentiation, mice genetically disrupted for the sept4 locus were generated via homologous recombination of the targeting vector as shown in FIG. 12A. Correct targeting of the sept4 locus was confirmed by Southern blot analysis (FIG. 12B). Utilizing the construct as indicated resulted in deletion of exons 2-12 of the sept4 locus, resulting in the loss of all sept4 splice variants (FIG. 13).

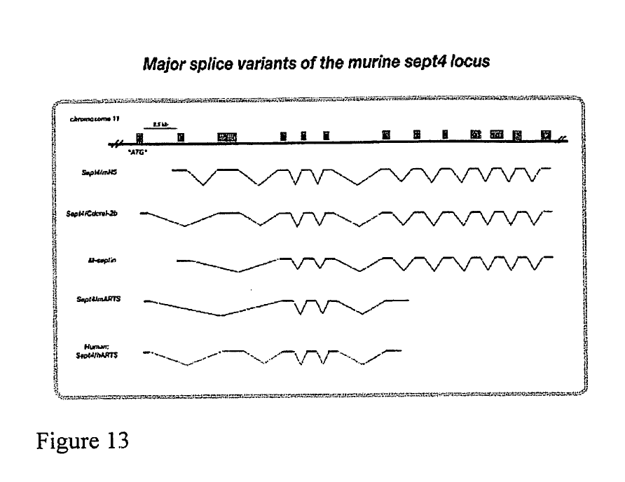

Histological evaluation of the testes of null mutants was conducted (FIG. 14). No histologic defects were observed in meiotic and post-meiotic cells in adult males (FIG. 14A, B). Not only was there no defect observed in pre- and post-meiotic stages of spermatogenesis, but mature sperm produced were comparable, in terms of histologic appearance by light microscopy, and in terms of the overall number of mature sperm produced and stored in the epididymis of the null mutants (FIG. 14C).

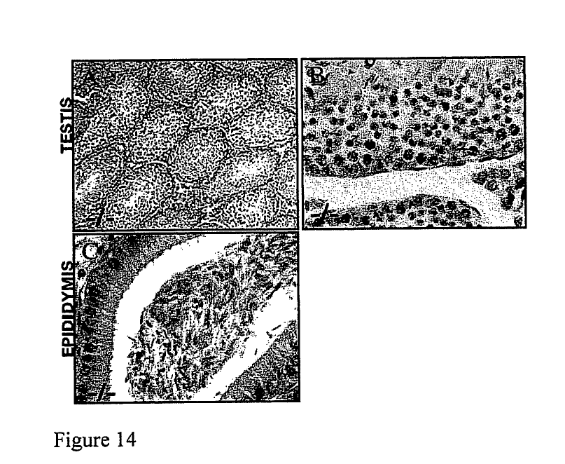

Following passage through the epididymis, however, structural defects in sperm of the sept4 mutants became apparent (FIG. 15). 180° bent sperm tails were among the structural defects demonstrated in sept4 null mutants, with bent sperm first appearing in the corpus (FIG. 15F). Sperm isolated from control mice exhibited no structural defects (FIG. 15C), in marked comparison.

### Example 12

### Sept4 Encoded Proteins are Essential for Formation of the Sperm Annulus

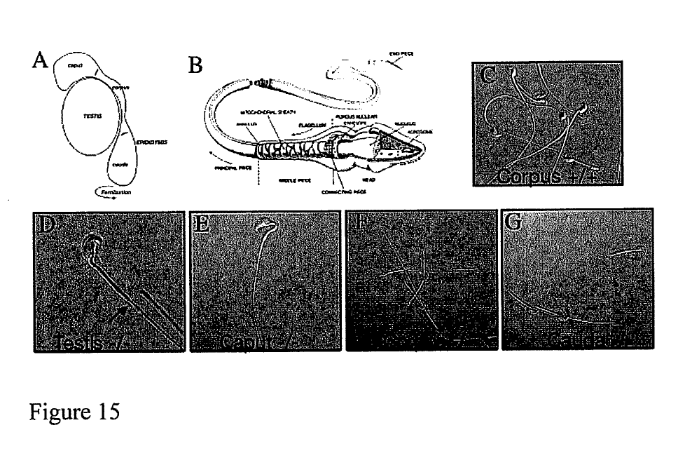

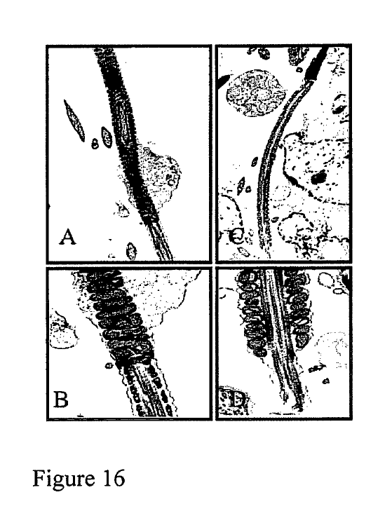

To further identify the structural defects seen in sept4 null mutants, transmission electron microscopy of sperm isolated from the cauda of null mutants was conducted (FIG. 16). Sept4 null mutant sperm demonstrably lacked an annulus (FIG. 16, A, B), which led to their complete immobility. Sept4 encoded proteins were critical in the establishment of the annulus, whose absence result in production of bent sperm (FIG. 17). In the absence of an annulus in the sept4 null mutants, more than 90% of the sperm isolated from the cauda of these mice are 180° bent, in comparison to controls.

Thus Sept4 plays a demonstrable role in annulus formation in sperm, whose absence produces structurally mutated sperm incapable of motility, leading therefore to profound male infertility.

### Example 13

### Differential Caspase Activation During Spermatogenesis

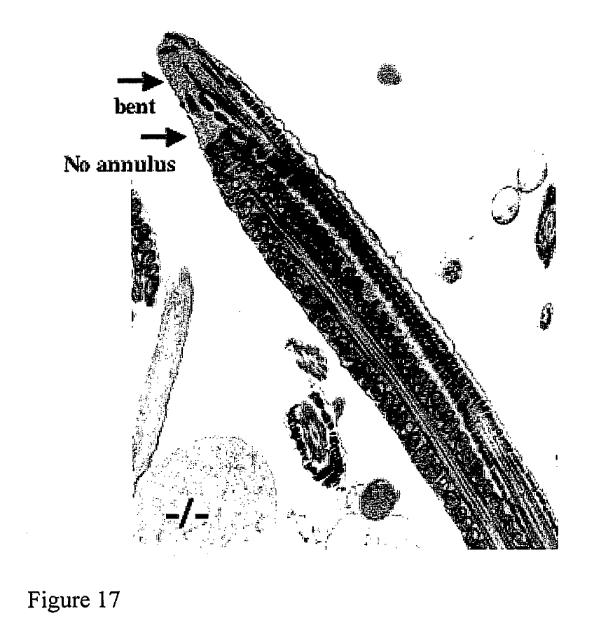

In order to further identify the role of caspases during spermatogenesis specific caspase activation and localization within sperm sub-cellular compartments was evaluated. Paraffin sections of wild-type testes were prepared for histologic evaluation, and probed for CM1 staining, indicating caspase 3 expression Caspase 3 was found in cytoplasmic droplets in spermatids in the lumen of the seminiferous tubules in wild-type adult fertile males (FIG. 18), indicating its role in late stage mammalian spermatogenesis.

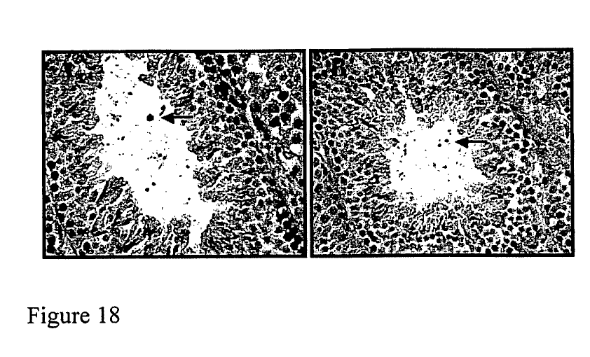

Caspase 3 activity is moreover, localized predominantly to the cytoplasmic droplet and head region of spermatids during late stages of spermatid differentiation (FIG. 19A-C). Mature spermatozoa demonstrate caspase 3 activity in cytoplasmic droplets and acrosomes (FIG. 19D-F).

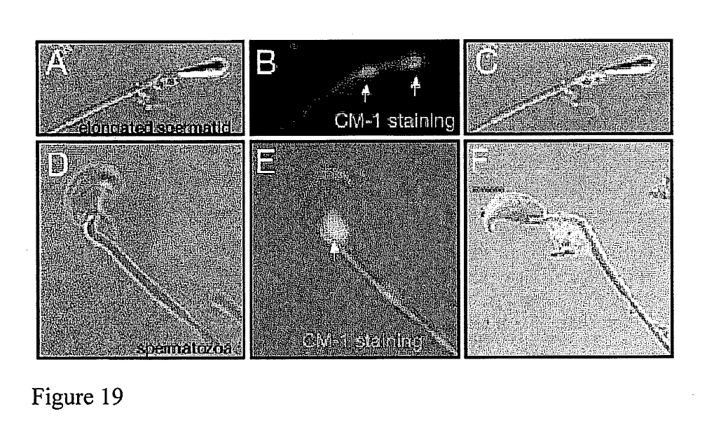

While caspase 3 activation is restricted to cytoplasmic droplets and head regions/acrosomes in spermatids and mature spermatozoa, respectively, caspase 9 activity is restricted to the principal piece of the sperm tail in epididymal cauda sperm (FIG. 20A). Similarly, caspase 6 activity is restricted to the principal piece of the sperm tail (FIG. 20C), which essentially overlaps with caspase 9 activity localization (FIG. 20D).

Thus a differential expression pattern for caspase activation is demonstrated, with caspase 3 activity correlated with earlier events in spermatid differentiation, and caspase 6 and 9 activity participating in later stages of sperm differentiation.

1. A method of inhibiting or abrogating spermatid differentiation, comprising the step of contacting a spermatogenic cell with an agent that inhibits or abrogates caspase expression, activity or function in said cell, thereby inhibiting or abrogating spermatid differentiation.

2. The method of claim 1 wherein said spermatogenic cell is a spermatogonium, a spermatocyte or a spermatid.

3. The method of claim 1, wherein said agent is Z-VAD, Cbz-Pro-Asp-fmk, Cbz-Thz-Asp-fmk, or 4-ClCbz-Val-Asp-fmk.

4. The method of claim 1, wherein said caspase is caspase 3, 6 or 9.

5. A method of inhibiting or abrogating spermatid differentiation, comprising the step of contacting a spermatogenic cell with an agent that inhibits or abrogates cyt-c-d expression or activity in said cell, thereby inhibiting or abrogating spermatid differentiation.

6. The method of claim 5, wherein said spermatogenic cell is a spermatogonium, a spermatocyte or a spermatid

7. The method of claim 5, wherein inhibiting or abrogating cyt-c-d expression or activity in said cell, results in inhibition or abrogation of caspase expression, activity and/or activation.

8. A method of inhibiting or abrogating mature sperm formation, comprising the step of contacting a spermatogenic cell with an agent that inhibits or abrogates Sept4/ARTS expression or activity in said cell, thereby inhibiting or abrogating mature sperm formation.

9. The method of claim 8, wherein said spermatogenic cell is a spermatogonium, a spermatocyte or a spermatid.

10. The method of claim 8, wherein inhibiting or abrogating Sept4/ARTS expression or activity in said cell, results in inhibition or abrogation of caspase expression, activity and/or activation.

11. The method of claim 8, wherein inhibiting or abrogating Sept4/ARTS expression or activity in said cell, results in diminished or absent annulus formation.

12. The method of claim 8, wherein inhibiting or abrogating Sept4/ARTS expression or activity in said cell, results in the production of bent sperm.

13. A method of inhibiting or abrogating spermatid differentiation, comprising the step of contacting a spermatogenic cell with an agent that inhibits or abrogates Bruce expression or activity in said cell, thereby inhibiting or abrogating spermatid differentiation.

14. The method of claim 13, wherein said spermatogenic cell is a spermatogonium, a spermatocyte or a spermatid.

15. The method of claim 13, wherein inhibiting or abrogating Bruce expression or activity in said cell results in damage to the cells as a result of caspase activity.

16. A method of male contraception in a subject, comprising contacting a spermatogenic cell in said subject with an agent that diminishes or abrogates caspase expression or activity in said spermatogenic cell.

17. The method of claim 16, wherein said spermatogenic cell is a spermatogonium, a spermatocyte or a spermatid.

18. The method of claim 16, wherein said agent is Z-VAD Cbz-Pro-Asp-fmk, Cbz-Thz-Asp-fmk, or 4-ClCbz-Val-Asp-fmk.

19. A method of male contraception in a subject, comprising contacting a spermatogenic cell in said subject with an agent that diminishes or abrogates cyt-c-d expression or activity in said spermatogenic cell.

20. The method of claim 19 wherein said spermatogenic cell is a spermatogonium, a spermatocyte or a spermatid.

21. The method of claim 19, wherein inhibiting or abrogating cyt-c-d expression or activity in said cell, results in inhibition or abrogation of caspase expression, activity and/or activation.

22. A method of male contraception in a subject, comprising contacting a spermatogenic cell in said subject with an agent that diminishes or abrogates Sept4/ARTS expression or activity in said spermatogenic cell.

23. The method of claim 22 wherein said spermatogenic cell is a spermatogonium, a spermatocyte or a spermatid.

24. The method of claim 22, wherein inhibiting or abrogating Sept4/ARTS expression or activity in said cell, results in inhibition or abrogation of caspase expression, activity and/or activation.

25. The method of claim 22, wherein inhibiting or abrogating Sept4/ARTS expression or activity in said cell, results in inhibition or abrogation of annulus formation.

26. The method of claim 22, wherein inhibiting or abrogating Sept4/ARTS expression or activity in said cell, results in the formation of bent sperm.

27. A method of male contraception in a subject, comprising contacting a spermatogenic cell in said subject with an agent that diminishes or abrogates Bruce expression or activity in said spermatogenic cell.

28. The method of claim 27 wherein said spermatogenic cell is a spermatogonium, a spermatocyte or a spermatid.

29. The method of claim 27, wherein inhibiting or abrogating Bruce expression or activity in said cell results in damage to the cells as a result of caspase activity.

30. A method of arthropod pest control, comprising, contacting a spermatogenic cell of said pest with an agent that diminishes or abrogates caspase expression or activity.

31. The method of claim 30 wherein said spermatogenic cell is a spermatogonium, a spermatocyte or a spermatid.

32. The method of claim 30, wherein said agent is Z-VAD, Cbz-Pro-Asp-fmk, Cbz-Thz-Asp-fmk, or 4-ClCbz-Val-Asp-fmk.

33. The method of claim 30, wherein said arthropod is an insect.

34. A method of arthropod pest control, comprising, contacting a spermatogenic cell of said pest with an agent that diminishes or abrogates cyt-c-d expression or activity.

35. The method of claim 34, wherein said spermatogenic cell is a spermatogonium, a spermatocyte or a spermatid.

36. The method of claim 34, wherein inhibiting or abrogating cyt-c-d expression or activity in said cell, results in inhibition or abrogation of caspase expression, activity and/or activation.

37. The method of claim 34, wherein said arthropod is an insect.

38. A method of arthropod pest control, comprising, contacting a spermatogenic cell of said pest with an agent that diminishes or abrogates Sept4/ARTS expression or activity.

39. The method of claim 38 wherein said spermatogenic cell is a spermatogonium, a spermatocyte or a spermatid.

40. The method of claim 38, wherein inhibiting or abrogating Sept4/ARTS expression or activity in said cell, results in inhibition or abrogation of caspase expression, activity and/or activation.

41. The method of claim 38, wherein inhibiting or abrogating Sept4/ARTS expression or activity in said cell, results in inhibition or abrogation of annulus formation.

42. The method of claim 38, wherein inhibiting or abrogating Sept4/ARTS expression or activity in said cell, results in the formation of bent sperm.

43. The method of claim 38, wherein said arthropod is an insect.

44. A method of arthropod pest control, comprising, contacting a spermatogenic cell of said pest with an agent that diminishes or abrogates Bruce expression or activity.

45. The method of claim 44 wherein said spermatogenic cell is a spermatogonium, a spermatocyte or a spermatid.

46. The method of claim 44, wherein inhibiting or abrogating Bruce expression or activity in said cell, results in damage to the cell as a result of caspase activity.

47. The method of claim 44, wherein said arthropod is an insect.

48. A method of stimulating or enhancing spermatid differentiation, comprising the step of contacting a spermatogenic cell with an agent that stimulates or enhances caspase expression or activity in said cell, thereby stimulating or enhancing spermatid differentiation.

49. The method of claim 48 wherein said spermatogenic cell is a spermatogonium, a spermatocyte or a spermatid.

50. A method of stimulating or enhancing spermatid differentiation, comprising the step of contacting a spermatogenic cell with an agent that stimulates or enhances cyt-c-d expression or activity in said cell, thereby stimulating or enhancing spermatid differentiation.

51. The method of claim 50, wherein said spermatogenic cell is a spermatogonium, a spermatocyte or a spermatid.

52. The method of claim 50, wherein stimulating or enhancing cyt-c-d expression or activity in said cell, results in stimulation or enhancement of caspase expression, activity and/or activation.

53. A method of stimulating or enhancing spermatid differentiation, comprising the step of contacting a spermatogenic cell with an agent that stimulates or enhances Sept4/ARTS expression or activity in said cell, thereby stimulating or enhancing spermatid differentiation.

54. The method of claim 53, wherein said spermatogenic cell is a spermatogonium, a spermatocyte or a spermatid.

55. The method of claim 53, wherein stimulating or enhancing Sept4/ARTS expression or activity in said cell, results in stimulation or enhancement of caspase expression, activity and/or activation.

56. A method of stimulating or enhancing spermatid differentiation, comprising the step of contacting a spermatogenic cell with an agent that stimulates or enhances Bruce expression or activity in said cell, thereby stimulating or enhancing spermatid differentiation.

57. The method of claim 56, wherein said spermatogenic cell is a spermatogonium, a spermatocyte or a spermatid.

58. The method of claim 56, wherein stimulating or enhancing Bruce expression or activity in said cell, results in protection from caspase activity.

59. A method of treatment of infertility in a male subject in need thereof, comprising contacting a spermatogenic cell in said subject with an agent that stimulates or enhances caspase expression or activity in said cell.

60. The method of claim 59 wherein said spermatogenic cell is a spermatogonium, a spermatocyte or a spermatid.

61. A method of treatment of infertility in a male subject in need thereof, comprising contacting a spermatogenic cell in said subject with an agent that stimulates or enhances cytochrome c-d expression or activity in said cell.

62. The method of claim 61, wherein said spermatogenic cell is a spermatogonium, a spermatocyte or a spermatid.

63. The method of claim 61, wherein stimulating or enhancing cyt-c-d expression or activity in said cell, results in stimulation or enhancement of caspase expression, activity and/or activation.

64. A method of treatment of infertility in a male subject in need thereof, comprising contacting a spermatogenic cell in said subject with an agent that stimulates or enhances Sept4/ARTS expression or activity in said cell.

65. The method of claim 64, wherein said spermatogenic cell is a spermatogonium, a spermatocyte or a spermatid.

66. The method of claim 64, wherein stimulating or enhancing Sept4/ARTS expression or activity in said cell, results in stimulation or enhancement of caspase expression, activity and/or activation.

67. A method of treatment of infertility in a male subject in need thereof, comprising contacting a spermatogenic cell in said subject with an agent that stimulates or enhances Bruce expression or activity in said cell.

68. The method of claim 67, wherein said spermatogenic cell is a spermatogonium, a spermatocyte or a spermatid.

69. The method of claim 67, wherein stimulating or enhancing Bruce expression or activity in said cell, results in protection from caspase activity.

70. A method of identifying an agent that stimulates or increases the fertility rate of a male mammal comprising:
(a) administering a candidate agent to a non-human male transgenic animal genetically disrupted for caspase, cyt-c-d, Sept4/ARTS, or Bruce gene function; and
(b) determining the effect of said agent on a fertility rate of said non-human male transgenic animal, wherein an increased fertility rate indicates that the candidate agent increases the fertility rate of a mammal.

71. A method of identifying an agent that decreases or abrogates the fertility rate of a male mammal comprising:
(a) administering a candidate agent to a non-human male animal expressing a caspase, cyt-c-d, Sept4/ARTS, or Bruce gene, wherein said agent alters said caspase, cyt-c-d, Sept4/ARTS, or Bruce gene or gene product expression or activity; and
(b) determining the effect of said agent on a fertility rate of said non-human male animal,
wherein a decreased fertility rate indicates that the candidate agent decreases or abrogates the fertility rate of a mammal.

72. A method of identifying an agent that decreases or abrogates the fertility rate of a male arthropod comprising:
(a) administering a candidate agent to a male arthropod expressing a caspase, cyt-c-d, Sept4/ARTS, or Bruce gene, wherein said agent alters said caspase, cyt-c-d, Sept4/ARTS, or Bruce gene or gene product expression or activity; and
(b) determining the effect of said agent on a fertility rate of said male arthropod,
wherein a decreased fertility rate indicates that the candidate agent decreases or abrogates the fertility rate of a male arthropod.

73. A method of monitoring in vivo caspase inhibition in a male subject in need, comprising the steps of:
(a) obtaining a semen sample from a male subject in need; and
(b) determining the presence of spermatid differentiation in said semen sample,
wherein diminished spermatid differentiation indicates in vivo caspase inhibition.

74. A transgenic animal whose genome comprises a disruption of the cyt-c-d gene, such that the animal lacks or has reduced levels of functional cyt-c-d protein, and wherein said animal exhibits decreased or abrogated male fertility or decreased or abrogated caspase activity.

75. Cultured cells isolated from the animal of claim 74, wherein the genomes of said cells comprise a disruption of the cyt-c-d gene and said cells exhibit decreased or abrogated male fertility or decreased or abrogated caspase activity.

76. A transgenic animal whose genome comprises a disruption of the Sept4/ARTS gene, such that the animal lacks or has reduced levels of functional Sept4/ARTS protein, and wherein said animal exhibits decreased or abrogated male fertility or decreased or abrogated caspase activity.

77. Cultured cells isolated from the animal of claim 76, wherein the genomes of said cells comprise a disruption of the Sept4/ARTS gene and said cells exhibit decreased or abrogated male fertility or decreased or abrogated caspase activity.

78. A transgenic animal whose genome comprises a disruption of the Bruce gene, such that the animal lacks or has reduced levels of functional Bruce protein, and wherein said animal exhibits decreased or abrogated male fertility or decreased or abrogated spermatogenesis.

79. Cultured cells isolated from the animal of claim 67, wherein the genomes of said cells comprise a disruption of the Bruce gene and said cells exhibit decreased or abrogated male fertility or decreased or abrogated spermatogenesis.
